(19) United States
(12) Patent Application Publication
(10) Pub. No.: US 2008/0161400 A1
(43) Pub. Date: Jul. 3, 2008

(54) USE OF FORMS OF PROPOFOL FOR TREATING DISEASES ASSOCIATED WITH OXIDATIVE STRESS

(75) Inventors: Peter Virsik, Portola Valley, CA (US); Feng Xu, Palo Alto, CA (US)

Correspondence Address: Timothy A. Worrall, Ph.D., J.D.;Dorsey & Whitney LLP, Suite 300, 500 California Street, San Francisco, CA, 94104-1513, US

(73) Assignee: XenoPort, Inc.

(21) Appl. No.: 11/923,444

(22) Filed: Oct. 24, 2007

Related U.S. Application Data
(60) Provisional application No. 60/854,868, filed on Oct. 26, 2006.

(51) Int. Cl. A61K 31/195; A61K 31/05; A61P 9/00; A61P 25/00; A61P 11/00; A61P 1/16

(52) U.S. Cl. 514/567; 514/731

(57) ABSTRACT

Methods of treating diseases associated with oxidative stress such as metabolic diseases, cardiovascular diseases, neurological diseases, liver diseases, and pulmonary diseases in a patient comprising orally administering a therapeutically effective amount of forms of propofol that provide a high oral bioavailability of propofol are disclosed.

This application claims benefit of U.S. Provisional Application No. 60/854,868 filed Oct. 26, 2006, which is incorporated by reference herein in its entirety.

### FIELD

Disclosed herein are methods of treating diseases associated with oxidative stress such as metabolic diseases, cardiovascular diseases, neurological diseases, liver diseases, and pulmonary diseases in a patient comprising orally administering a therapeutically effective amount of forms of propofol that provide a high oral bioavailability of propofol.

### BACKGROUND

Increased oxidative stress is implicated in the pathology of a variety of diseases including metabolic, cardiovascular, neurological, liver, and pulmonary diseases. Oxidative stress is defined in general as excess formation and/or insufficient removal of highly reactive molecules such as reactive oxygen species (ROS) and reactive nitrogen species (RNS) (Maritim et al., J Biochem Mol Toxicol 2003, 17(1), 24-38; and Yorek, Free Radical Research 2003, 37(5), 471-480). ROS include free radicals such as superoxide (*O₂⁻), hydroxyl (*OH), peroxyl (*RO₂), hydroperoxyl (*HRO₂⁻) as well as nonradical species such as hydrogen peroxide (H₂O₂) and hydrochlorous acid (HOCl). ROS are continuously produced during normal physiologic processes, and are removed by the activity of antioxidant enzymes such as glutathione peroxidase, catalase, and superoxide dismutase. Under pathological conditions, ROS can be overproduced and result in oxidative stress. RNS include free radicals such as nitric oxide (*NO) and nitrogen dioxide (*NO₂⁻) as well as nonradicals such as peroxynitrite (ONOO⁻), nitrous oxide (HNO₂), and alkyl peroxynitrates (RONOO). *NO₂ is normally produced from L-arginine by NO synthase (NOS). Three isoforms have been identified from three distinct genes: neuronal NOS (nNOS), inducible NOS (iNOS), and endothelial NOS (eNOS). In the vascular endothelium, *NO mediates vasorelaxation by its acting on guanylate cyclase in vascular smooth muscle cells, initiating a cascade that leads to vasorelaxation. *NO also displays anti-proliferative properties and inhibits platelet and leukocyte adhesion to vascular endothelium. However, *NO easily reacts with superoxide (*O₂⁻), generating the highly reactive molecule ONOO⁻ and triggering a cascade of harmful effects.

Exogenous compounds can protect against oxidative stress by acting as direct chain-breaking antioxidants or free radical scavengers, inhibiting ROS and RNS formation, chelating transition metals, and inducing enzymes involved in detoxification and damage repair. Administration of antioxidants such as α-tocopherol, butylated hydroxytoluene (BHT), butylated hydroxyanisole (BHA), propyl gallate, and tert-butylhydroquinone to neutralize ROS and RON has met with variable success.

The antioxidant properties of propofol are well known. Propofol (2,6-diisopropylphenol),

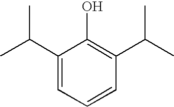

is a low molecular weight phenol that is widely used as an intravenous sedative-hypnotic agent in the induction and maintenance of anesthesia and/or sedation in mammals. The advantages of propofol as an anesthetic include rapid onset of anesthesia, rapid clearance, and minimal side effects (Langley et al., Drugs 1988, 35, 334-372). The hypnotic effects of propofol may be mediated through interaction with the GABA_{A} receptor complex, a hetero-oligomeric ligand-gated chloride ion channel (Peduto et al., Anesthesiology 1991, 75, 1000-1009). Propofol also has a broad range of other biological and medical applications, which are evident at sub-anesthetic (e.g., sub-hypnotic) and sub-sedative doses. Propofol prevents lipid peroxidation, inhibits radical chain reactions, and exhibits antioxidant capacity against various antioxidant systems in vitro is attributed to its activity as a strong lipid peroxidation inhibitor, reducing agent, metal chelator, hydrogen donating ability and effectiveness in scavenging hydrogen peroxide, superoxide, and free radicals (Gulcin et al., Chem Pharm Bull 2005, 53(3), 281-285). Antioxidant effects of propofol include decreased cerebral metabolic rate for oxygen and cerebral metabolic rate of glucose, inhibition of neutrophil respiratory burst, inhibition of mitochondrial permeability transition, scavenge reactive oxygen species, decreased glutamate efflux, inhibition of NMDA receptor activity, and enhanced glutamate reuptake (Wilson and Gelb, J Neurosurgical Anesthesiology 2002, 14(1), 66-79). Propofol has been shown to have specific in vivo activity in attenuating the overproduction of NO and O₂*⁻ of vascular endothelial cells (Yu et al., Crit. Care Med 2006, 34(2), 453-60) and exhibits neuroprotective effects on neuronal cell death induced by ¹O₂* (Heyne et al., Biochemica Biophysica Acta 2005, 1724, 100-107). Propofol (2,6-diisopropylphenol) is shown to have more effective in vitro antioxidant capacity than commonly used antioxidants having similar structure such as butylated hydroxyanisole (BHA), butylated hydroxytoluene (BHT), propyl gallate, and tert-butylhydroquinone (Aarts et al., FEBS Letts 1995, 357, 83-85; Gülcin et al., Chem Pharm Bull 2005, 53(3), 281-285; and Boisset et al., Arch Toxicol 2004, 78(11), 635-42). Studies suggest that antioxidants capable of operating intracellularly are more effective in addressing the consequences of oxidative stress. In this regard, propofol, which is readily soluble in biomembranes and is shown to accumulate in biomembranes more readily than other antioxidants such as vitamin E, may be more effective in enhancing antioxidant defense of tissues and specifically lipophilic membrane environments (Murphy et al., Eur J Anaesthesiol 1993, 10, 261-266).

Propofol is rapidly metabolized in mammals with the drug being eliminated predominantly as glucuronidated and sulfated conjugates of propofol and 4-hydroxypropofol (Langley et al., Drugs 1988, 35, 334-372). Propofol is poorly absorbed in the gastrointestinal tract and only from the small intestine. When orally administered as a homogeneous liquid suspension, propofol exhibits an oral bioavailability of less than 5% that of an equivalent intravenous dose of propofol. Propofol clearance exceeds liver blood flow, which indicates that extrahepatic tissues contribute to the overall metabolism of the drug. Human intestinal mucosa glucuronidates propofol in vitro and oral dosing studies in rats indicate that approximately 90% of the administered drug undergoes first pass metabolism, with extraction by the intestinal mucosa accounting for the bulk of this pre-systemic elimination (Raoof et al., Pharm. Res. 1996, 13, 891-895). Because of its poor oral bioavailability and extensive first-pass metabolism, propofol is administered by injection or intravenous infusion and oral administration of propofol has not been considered therapeutically effective. This has prevented investigations into the efficacy of propofol for treating chronic pathologies and diseases or conditions for which intravenous infusion is not appropriate. Recently, several methods for improving propofol absorption from the gastrointestinal tract and/or minimizing first pass metabolism have been demonstrated.

For example, propofol prodrugs that exhibit enhanced oral bioavailability and that are sufficiently labile under physiological conditions to provide therapeutically effective concentrations of propofol following oral administration have been described Gallop et al., U.S. Pat. Nos. 7,220,875 and 7,230,003; and Xu et al., U.S. Application Publication Nos. 2006/0041011, and 2006/0205969, and U.S. patent application Ser. No. 11/180,064, each of which is incorporated by reference herein in its entirety. These propofol prodrugs provide enhanced oral bioavailability of propofol and can also facilitate oral propofol regimens capable of providing sustained therapeutically effective concentrations of propofol appropriate for treating chronic diseases and disorders. The availability of forms of propofol that provide a high oral bioavailability of propofol, such as the propofol prodrugs disclosed by Gallop et al. and by Xu et al. enable the use of such forms of propofol for treating diseases where it is desirable to administer propofol orally.

### SUMMARY

Accordingly, methods of treating a disease associated with oxidative stress in a patient comprising orally administering to a patient in need of such treatment a therapeutically effective amount of at least one form propofol that is capable of providing a high oral bioavailability of propofol.

This and other features of the present disclosure are set forth herein.

### BRIEF DESCRIPTION OF THE DRAWINGS

The skilled artisan will understand that the drawings, described herein, are for illustration purposes only. The drawings are not intended to limit the scope of the present disclosure.

FIG. 1 shows propofol blood concentrations following oral administration of compound (2) to rats at doses from 25 mg-equivalent/kg to 300 mg-equivalent/kg.

FIG. 2 shows propofol blood concentrations following oral administration of compound (2) to rats at doses from 400 mg-equivalent/kg to 800 mg-equivalent/kg.

FIG. 3 shows propofol blood concentrations following oral administration of compound (2) to dogs at doses from 50 mg-equivalent/kg to 150 mg-equivalent/kg.

### DETAILED DESCRIPTION

### Definitions

A dash (“-”) that is not between two letters or symbols is used to indicate a point of attachment for a substituent. For example, —CONH₂ is attached through the carbon atom.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group comprises from 1 to 20 carbon atoms, in certain embodiments, from 1 to 10 carbon atoms, and in certain embodiments, from 1 to 8 or 1 to 6 carbon atoms.

“Acyl” by itself or as part of another substituent refers to a radical —C(O)R⁷⁰, where R⁷⁰ is hydrogen, alkyl, heteroalkyl, cycloalkyl, cycloheteroalkyl, cycloalkylalkyl, cycloheteroalkylalkyl, aryl, heteroaryl, arylalkyl, or heteroarylalkyl, which can be substituted, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR⁷¹ where R⁷¹ is alkyl, cycloalkyl, cycloalkylalkyl, aryl, or arylalkyl, which can be substituted, as defined herein. In some embodiments, alkoxy groups have from 1 to 8 carbon atoms. Examples of alkoxy groups include, but are not limited to, methoxy, ethoxy, propoxy, butoxy, cyclohexyloxy, and the like.

“Alkoxycarbonyl” by itself or as part of another substituent refers to a radical —C(O)OR⁷² where R⁷² represents an alkyl, as defined herein. Examples of alkoxycarbonyl groups include, but are not limited to, methoxycarbonyl, ethoxycarbonyl, propoxycarbonyl, and butoxycarbonyl, and the like.

“Amino” refers to the radical —NH₂.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocylic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can comprise from 5 to 20 carbon atoms, and in certain embodiments, from 5 to 12 carbon atoms. Aryl, however, does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound in a biological fluid in a patient as a function of time following administration of the compound to the patient. Examples of biological fluids include plasma and blood. The AUC can be determined by measuring the concentration of a compound in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the disclosure herein, an AUC for propofol can be determined by measuring the concentration of propofol in the plasma or blood of a patient following oral administration of a dosage form comprising a form of propofol, such as a propofol prodrug or a propofol tight-ion pair complex.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to peak concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the highest drug concentration observed in the plasma or blood following a dose of drug.

Compounds encompassed by structural Formulae (I)-(IV) disclosed herein include any specific compounds within these formulae. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

Compounds of Formulae (I)-(IV) include, but are not limited to, optical isomers of compounds of Formulae (I)-(IV), racemates thereof, and other mixtures thereof. In such embodiments, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formulae (I)-(IV) include Z- and E-forms (e.g., cis- and trans-forms) of compounds with double bonds. In embodiments in which compounds of Formulae (I)-(IV) exist in various tautomeric forms, compounds of the present disclosure include all tautomeric forms of the compound.

The compounds of Formulae (I)-(IV) may also exist in several tautomeric forms including the enol form, the keto form, and mixtures thereof. Accordingly, the chemical structures depicted herein encompass all possible tautomeric forms of the illustrated compounds. The compounds of Formulae (I)-(IV) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated, or N-oxides. Certain compounds may exist in single or multiple crystalline or amorphous forms. In general, all physical forms are equivalent for the uses contemplated herein and are intended to be within the scope of the present disclosure.

Further, when partial structures of the compounds are illustrated, an asterisk (*) indicates the point of attachment of the partial structure to the rest of the molecule.

“Cycloalkoxycarbonyl” by itself or as part of another substituent refers to a radical —C(O)OR⁷⁶ where R⁷⁶ represents an cycloalkyl group as defined herein. Examples of cycloalkoxycarbonyl groups include, but are not limited to, cyclobutyloxycarbonyl, cyclohexyloxycarbonyl, and the like.

“Cycloalkyl” by itself or as part of another substituent refers to a partially saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₃₋₁₂ cycloalkyl or C₅₋₁₂ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“Disease” refers to a disease, disorder, condition, or symptom.

“Dosage form” means a pharmaceutical composition in a medium, carrier, vehicle, or device suitable for administration to a patient.

“Halogen” refers to a fluoro, chloro, bromo, or iodo group.

“Heteroalkyl” by itself or as part of another substituent refer to an alkyl group in which one or more of the carbon atoms (and any associated hydrogen atoms) are independently replaced with the same or different heteroatomic groups. In some embodiments, heteroalkyl groups have from 1 to 8 carbon atoms. Examples of heteroatomic groups include, but are not limited to, —O—, —S—, —O—O—, —S—S—, —O—S—, —NR⁷⁷R⁷⁸—, ═N—N═, —N═N—, —N═N—NR⁷⁹R⁸⁰, —PR⁸¹—, —P(O)₂—, —POR⁸², —O—P(O)₂—, —SO—, —SO₂—, —SnR⁸³R⁸⁴— and the like, where R⁷⁷, R⁷⁸, R⁷⁹, R⁸⁰, R⁸¹, R⁸², R⁸³, and R⁸⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, or substituted heteroarylalkyl. Where a specific level of saturation is intended, the nomenclature “heteroalkanyl,” “heteroalkenyl,” or “heteroalkynyl” is used.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which can be aromatic or non-aromatic in which at least one ring atom is a heteroatom. Heteroaryl encompasses 5- to 12-membered aromatic, such as 5- to 7-membered, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“Heterocycloalkylalkyl” by itself or as part of another substituent refers to an acyclic alkyl radical in which one of the hydrogen atoms bonded to a carbon atom, typically a terminal or sp³ carbon atom, is replaced with a heterocycloalkyl group. Where specific alkyl moieties are intended, the nomenclature heterocycloalkylalkanyl, heterocycloalkylalkenyl, or heterocycloalkylalkynyl is used. In certain embodiments, a heterocycloalkylalkyl group is a 6- to 30-membered heterocycloalkylalkyl, e.g., the alkanyl, alkenyl, or alkynyl moiety of the heterocycloalkylalkyl is 1- to 10-membered and the heterocycloalkyl moiety is a 5- to 20-membered heterocycloalkyl, and in certain embodiments, 6- to 20-membered heterocycloalkylalkyl, e.g., the alkanyl, alkenyl, or alkynyl moiety of the heterocycloalkylalkyl is 1- to 8-membered and the heterocycloalkyl moiety is a 5- to 12-membered heterocycloalkyl.

“Forms of propofol” means a chemical entity comprising propofol that when orally administered to a patient provides a high oral bioavailability of propofol in the systemic circulation of the patient. A chemical entity that provides a high oral bioavailability of propofol comprises propofol bonded either covalently or non-covalently to one or more moieties that facilitate absorption of the chemical entity and/or propofol from the gastrointestinal tract. In certain embodiments, a form of propofol that provides a high oral bioavailability of propofol comprises a propofol prodrug in which propofol is covalently bonded to at least one promoiety. In certain embodiments, a form of propofol that provides a high oral bioavailability of propofol comprises a complex in which propofol is non-covalently associated with at least one moiety. A form of propofol may release propofol in the gastroinstinal tract, during translocation across the intestinal lumen, in the systemic circulation, and/or intracellularly. In certain embodiments, a form of propofol that provides a high oral bioavailability of propofol may be absorbed from the gastrointestinal tract and enter the systemic circulation intact. In certain embodiments, the oral bioavailability of propofol is high when it is greater than about 10% that of an equivalent intravenous dose of propofol, in certain embodiments, when it is greater than about 20% that of an equivalent intravenous dose of propofol, in certain embodiments, when it is greater than about 40% that of an equivalent intravenous dose of propofol, in certain embodiments, when it is greater than about 60% that of an equivalent intravenous dose of propofol.

“Hydroxyl” refers to the group —OH.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” includes animals and mammals, such as for example, humans.

“Pharmaceutical composition” refers to at least one compound and a pharmaceutically acceptable vehicle with which the compound is administered to a patient.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound of the present disclosure can be administered to a patient and which does not destroy the pharmacological activity thereof and which is nontoxic when administered in doses sufficient to provide a therapeutically effective amount of the compound.

“Prodrug” refers to a derivative of a drug molecule that requires a transformation within the body to release the active drug. Prodrugs are frequently, although not necessarily, pharmacologically inactive until converted to the parent drug.

“Prodrug of propofol” refers to a compound in which a promoiety, which is cleavable in vivo, is covalently bound to the propofol molecule. In certain embodiments, a prodrug may be actively transported by transporters expressed in the enterocytes lining the gastrointestinal tract such as, for example, the PEPT1 transporter. Propofol prodrugs can be stable in the gastrointestinal tract and following absorption are cleaved in the systemic circulation to release propofol. In certain embodiments, a prodrug of propofol provides a greater oral bioavailability of propofol compared to the oral bioavailability of propofol when administered as a uniform liquid immediate release formulation. In certain embodiments, a prodrug of propofol provides a high oral bioavailability of propofol, or example, exhibiting a propofol oral bioavailability that is at least 10 times greater than the oral bioavailability of propofol when orally administered in an equivalent dosage form. In certain embodiments, a prodrug of propofol is a compound having a structure encompassed by any one of Formulae (I)-(IV), compound (1), and compound (2), infra. In certain embodiments, a propofol prodrug is compound (2), a pharmaceutically acceptable salt thereof, or a pharmaceutically acceptable solvate of any of the foregoing.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach or the agent may be supplied exogenously. For example, for a prodrug of Formula (IV), the drug is propofol and the promoiety has the structure:

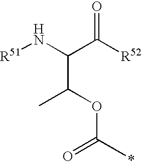

where R⁵¹ and R⁵² are as defined herein.

“Solvate” refers to a molecular complex of a compound with one or more solvent molecules in a stoichiometric or non-stoichiometric amount. Such solvent molecules are those commonly used in the pharmaceutical art, which are known to be innocuous to recipient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a complex where the one or more solvent molecules are water including monohydrates and hemi-hydrates.

“Substantially one diastereomer” refers to a compound containing two or more stereogenic centers such that the diastereomeric excess (d.e.) of the compound is greater than or about at least 90%. In certain embodiments, the d.e. is, for example, greater than or at least about 91%, about 92%, about 93%, about 94%, about 95%, about 96%, about 97%, about 98%, or about 99%.

“Substituted” refers to a group in which one or more hydrogen atoms are independently replaced with the same or different substituent(s). Examples of substituents include, but are not limited to, -Q, —R⁶⁰, —O⁻, (—OH), ═O, —OR⁶⁰, —SR⁶⁰, —S⁻, ═S, —NR⁶⁰R⁶¹, ═NR⁶⁰, —CX₃, —CN, —CF₃, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O⁻)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶⁰, C(O)O⁻, —C(S)OR⁶⁰, —NR⁶²C(O)NR⁶⁰R⁶¹, —NR⁶²C(S)NR⁶⁰R⁶⁰, —NR⁶²C(NR⁶³)NR⁶⁰R⁶¹, —C(NR⁶²)NR⁶⁰R⁶¹, —S(O)₂, NR⁶⁰R⁶¹, —NR⁶³S(O)₂R⁶¹, —NR⁶¹C(O)R⁶⁰, and —S(O)R⁶⁰ where each Q is independently a halogen; each R⁶⁰ and R⁶¹ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl, substituted heteroaryl, arylalkyl, substituted arylalkyl, heteroarylalkyl, or substituted heteroarylalkyl, or R⁶⁰ and R⁶¹ together with the nitrogen atom to which they are bonded form a cycloheteroalkyl, substituted cycloheteroalkyl, heteroaryl, or substituted heteroaryl ring, and R⁶² and R⁶³ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, or substituted heteroarylalkyl, or R⁶² and R⁶³ together with the atom to which they are bonded form one or more cycloheteroalkyl, substituted cycloheteroalkyl, heteroaryl, or substituted heteroaryl rings. In certain embodiments, a tertiary amine or aromatic nitrogen may be substituted with one or more oxygen atoms to form the corresponding nitrogen oxide.

In certain embodiments, substituted aryl and substituted heteroaryl include one or more of the following substitute groups: F, Cl, Br, C₁₋₃ alkyl, substituted alkyl, C₁₋₃ alkoxy, —S(O)₂NR⁶⁰R⁶¹, —NR⁶⁰R⁶¹, —CF₃, —OCF₃, —CN, —NR⁶⁰S(O)₂R⁶¹, —NR⁶⁰C(O)R⁶¹, C₅₋₁₀ aryl, substituted C₅₋₁₀ aryl, C₅₋₁₀ heteroaryl, substituted C₅₋₁₀ heteroaryl, —C(O)OR⁶⁰, —NO₂, —C(O)R⁶⁰, —C(O)NR⁶⁰R⁶¹, —OCHF₂, C₁₋₃ acyl, —SR⁶⁰, —S(O)₂OH, —S(O)₂R⁶⁰, —S(O)R⁶⁰, —C(S)R⁶⁰, —C(O)O⁻, —C(S)OR⁶⁰, —NR⁶⁰C(O)NR⁶¹R⁶², —NR⁶⁰C(S)NR⁶¹R⁶², and —C(NR⁶⁰)NR⁶¹R⁶², C₃₋₈ cycloalkyl, and substituted C₃₋₈ cycloalkyl, wherein R⁶¹, R⁶², and R⁶² are each independently chosen from hydrogen and C₁₋₄ alkyl.

In certain embodiments, each substituent group can independently be chosen from halogen, —NO₂, —OH, —COOH, —NH₂, —CN, —CF₃, —OCF₃, C₁₋₈ alkyl, substituted C₁₋₈ alkyl, C₁₋₈ alkoxy, and substituted C₁₋₈ alkoxy.

“Controlled delivery” means continuous or discontinuous release of a drug over a prolonged period of time, wherein the drug is released at a controlled rate over a controlled period of time in a manner that provides for upper gastrointestinal and lower gastrointestinal tract delivery, coupled with improved drug absorption as compared to the absorption of the drug in an immediate release oral dosage form.

“Sustained release” refers to release of a therapeutic amount of a drug, a prodrug, or an active metabolite of a prodrug over a period of time that is longer than that of a conventional formulation of the drug, e.g. an immediate release formulation of the drug. For oral formulations, the term “sustained release” typically means release of the drug within the gastrointestinal tract lumen over a time period from about 2 to about 30 hours, and in certain embodiments, over a time period from about 4 to about 24 hours. Sustained release formulations achieve therapeutically effective concentrations of the drug in the systemic circulation over a prolonged period of time relative to that achieved by oral administration of a conventional formulation of the drug. “Delayed release” refers to release of a drug, a prodrug, or an active metabolite of a prodrug into the gastrointestinal lumen after a delayed time period, for example a delay of about 1 to about 12 hours, relative to that achieved by oral administration of a conventional formulation of the drug.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and to inhibiting at least one physical parameter which may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The “therapeutically effective amount” can vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

Reference is now made in detail to embodiments of the present disclosure. The disclosed embodiments are not intended to be limiting of the claims. To the contrary, the claims are intended to cover alternatives, modifications, and equivalents.

### Forms of Propofol

In certain embodiments, forms of propofol provide an oral bioavailability of propofol that is at least 10 times greater than the oral bioavailability of propofol when orally administered in an equivalent dosage form. In certain embodiments, forms of propofol provide an oral bioavailability of propofol that is at least 10 times greater than the oral bioavailability of propofol provided by propofol when orally administered to a patient as a uniform liquid immediate release formulation. Forms of propofol include prodrugs, conjugates, and complexes in which propofol is attached to at least one moiety. The moiety covalently or non-covalently attached to propofol may enhance permeability through gastrointestinal epithelia via passive and/or active transport mechanisms, may control the release of propofol in the gastrointestinal tract, and/or may inhibit enzymatic and chemical degradation of propofol in the gastrointestinal tract. For forms of propofol in which the moiety remains attached to the propofol molecule after absorption, the moiety may enhance permeability through other biological membranes, and/or can inhibit enzymatic and chemical degradation of propofol in the systemic circulation.

Reducing the rate of metabolism of a drug in the gastrointestinal tract and/or enhancing the rate by which a drug is absorbed from the gastrointestinal tract may enhance the oral bioavailability of a drug. An orally administered drug will pass through the gastrointestinal system in about 11 to 31 hours. In general, an orally ingested drug resides about 1 to 6 hours in the stomach, about 2 to 7 hours in the small intestine, and about 8 to 18 hours in the colon. The oral bioavailability of a particular drug will depend on a number of factors including the residence time in a particular region of the gastrointestinal tract, the rate the drug is metabolized within the gastrointestinal tract, the rate at which a drug is metabolized in the systemic circulation, and the rate by which the compound is absorbed from a particular region or regions of the gastrointestinal tract, which include passive and active transport mechanisms. Several methods have been developed to achieve these objectives, including drug modification, incorporating the drug or modified drug in a controlled release dosage form, and/or by co-administering adjuvants, which can be incorporated in the dosage form containing the active compound.

A drug may be modified to reduce the rate of drug metabolism in the gastrointestinal tract and/or to enhance and/or modify the absorption of the drug from the gastrointestinal tract. Forms of propofol that provide a high oral bioavailability of propofol include propofol tight-ion pairs and propofol prodrugs.

Wong et al., U.S. Application Publication No. 2005/0163850 (which is incorporated by reference herein in its entirety) forming tight-ion pair complexes of generally hydrophobic compounds such as alkyl sulfates or fatty acids. At physiologic pH in an aqueous environment, tight-ion pairs are not readily interchangeable with other loosely paired or free ions that may be present in the environment of the tight-ion pair. The tight-ion pair complexes disclosed by Wong et al. are characterized by a generally hydrophobic exterior and are intended to be more stable than loose ion pairs in the presence of water rendering the complexes more likely to move through intestinal epithelial membranes by paricellular or active transport. Such tight-ion pair complexes may enhance absorption of drugs as well as prodrugs in both the upper and lower gastrointestinal tract.

In certain embodiments, a form of propofol is a propofol prodrug. Examples of propofol prodrugs that provide a high oral bioavailability of propofol include bile acid prodrugs, peptide conjugates, and prodrugs in which propofol is bonded to an amino acid or small peptide via a linkage. Prodrugs are compounds in which a promoiety is typically covalently bonded to a drug. Following absorption from the gastrointestinal tract, the promoiety is cleaved to release the drug into the systemic circulation. While in the gastrointestinal tract, the promoiety can protect the drug from the harsh chemical environment, and can also facilitate absorption. Promoieties can be designed, for example, to enhance passive absorption, e.g., lipophilic promoieties, and/or enhance absorption via active transport mechanisms, e.g., substrate promoieties. In particular, active transporters differentially expressed in regions of the gastrointestinal tract may be preferentially targeted to enhance absorption. For example, a propofol prodrug may incorporate a promoiety that is a substrate of PEPT1 transporters expressed in the small intestine. Zerangue et al., U.S. Pat. No. 6,955,888 and U.S. Application Publication No. 2005/0214853 (each of which is incorporated by reference herein in its entirety) disclose methodologies for screening drugs, conjugates or conjugate moieties, linked or linkable to drugs, for their capacity to be transported as substrates via the PEPT1 and PEPT2 transporters, which are known to be expressed in the human small intestine (see, e.g., Fei et al., Nature 1964, 386, 563-566; Miyamoto et al., Biochimica et Biophysica Acta 1996, 1305, 34-38). Zerangue et al., U.S. Application Publication No. 2003/0158254 also disclose several transporters expressed in the human colon including the sodium dependent multi-vitamin transporter (SMVT) and monocarboxylate transporters MCT1 and MCT4, and methods of identifying agents, or conjugate moieties that are transporter substrates, and agents, conjugates, and conjugate moieties that may be screened for substrate activity. Zerangue et al. further disclose compounds that may be screened that are variants of known transporter substrates such as bile salts or acids, steroids, ecosanoids, or natural toxins or analogs thereof, as described by Smith, Am. J. Physiol 1987, 223, 974-978; Smith, Am J Physio. 1993, 252, G479-G484; Boyer, Proc Natl Acad Sci USA 1993, 90, 435-438; Fricker, Biochem J 1994, 299, 665-670; Ficker, Biochem J 1994, 299, 665-670; and Ballatori et al., Am J Physiol 2000, 278, G57-G63, and the linkage of drugs to conjugate moieties.

Conjugation to bile acids has been shown to enhance oral bioavailability of a drug. Bile acids are hydroxylated steroids that play a key role in digestion and absorption of fat and lipophilic vitamins. After synthesis in the liver, bile acids are secreted into bile and excreted by the gall bladder into the intestinal lumen where they emulsify and help solubilize lipophilic substances. Bile acids are conserved in the body by active uptake from the terminal ileum via the sodium-dependent transporter IBAT (or ASBT) and subsequent hepatic extraction by the transporter NTCP (or LBAT) located in the sinusoidal membrane of hepatocytes. Gallop et al. disclose prodrugs in which a drug is covalently attached to a cleavable linker, which in turn is covalently attached to a moiety, such as a bile acid or bile acid derivative that facilitates translocation of the conjugate across the intestinal epithelia via the bile acid transport system (see, Gallop et al., U.S. Pat. Nos. 6,984,634, 6,900,192, 6,984,634, 7,144,877, 7,053,076, and 7,049,305; and U.S. Application Publication Nos. 2005/0272710 and 2005/0288228, each of which is incorporated by reference herein in its entirety). Following absorption via the bile acid transport system, the linker is cleaved to release the drug into the systemic circulation.

Another drug-modification method for enhancing oral bioavailability includes covalent attachment of drugs directly to an amino acid or polypeptide that stabilizes the active agent, primarily in the stomach, through conformational protection (see, e.g., Piccariello et al., U.S. Pat. Nos. 6,716,452 and 7,060,708, and U.S. Application Publication No. 2004/0127397). Piccariello et al. disclose conjugates in which a drug, such as propofol, may be covalently attached directly to the N-terminus, the C-terminus or an amino acid side chain of a carrier peptide. In certain applications, the polypeptide may stabilize the drug in the gastrointestinal tract through conformational protection and/or act as a substrate for transporters such as PEPT transporters.

These prodrugs, which can provide enhanced oral bioavailability of propofol, are distinguishable from propofol prodrugs having promoieties that provide enhanced aqueous solubility of propofol for intravenous administration. Propofol exhibits poor aqueous solubility and it is desirable that intravenously administered drugs be water-soluble. Propofol is widely used as a hypnotic sedative for intravenous administration in the induction and maintenance of anesthesia or sedation in humans and animals. Propofol prodrugs with enhanced aqueous solubility for intravenous administration are disclosed, for example, by Stella et al., U.S. Pat. Nos. 6,204,257, 6,872,838, and 7,244,718; Marappan et al., U.S. Pat. No. 7,250,412; and Wingard et al., U.S. Application Publication No. 2005/0203068.

Examples of propofol prodrugs capable of providing an increased oral bioavailability of propofol in which propofol is bonded to an amino acid or small peptide via a linkage are disclosed in Gallop et al., U.S. Pat. Nos. 7,220,875 and 7,230,003; Xu et al., U.S. Application Publication No. 2006/0041011; Xu et al., Xu et al., U.S. Application Publication No. 2006/0205969, and U.S. patent application Ser. No. 11/180,064, each of which is incorporated by reference herein in its entirety.

In certain embodiments, prodrugs of propofol may be chosen from any of the genuses or species of compounds of Formula (I) as disclosed in Gallop et al., U.S. Pat. No. 7,220,875:

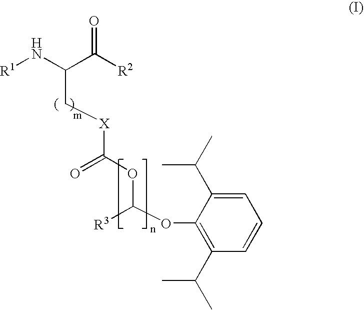

a pharmaceutically acceptable salt thereof, or a pharmaceutically acceptable solvate of any of the foregoing, wherein:

X is chosen from a bond, —CH₂—, —NR¹¹—, —O—, and —S—;

m is chosen from 1 and 2;

n is chosen from 0 and 1;

R¹ is chosen from hydrogen, [R⁵NH(CHR⁴)ₚC(O)]—, R⁶—, R⁶C(O)—, and R⁶OC(O)—;

R² is chosen from —OR⁷, and —[NR⁸(CHR⁹)_{q}C(O)OR⁷];

p and q are independently chosen from 1 and 2;

R³ is chosen from hydrogen, alkyl, substituted alkyl, alkoxycarbonyl, aryl, substituted aryl, arylalkyl, carbamoyl, substituted carbamoyl, cycloalkyl, substituted cycloalkyl, cycloheteroalkyl, heteroaryl, substituted heteroaryl, and heteroarylalkyl;

each R⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or, when R⁴ and R⁵ are attached to adjacent atoms then R⁴ and R⁵ together with the atoms to which they are bonded form a cycloheteroalkyl or substituted cycloheteroalkyl ring;

R⁵ is chosen from hydrogen, R⁶—, R⁶C(O)—, and R⁶OC(O)—;

R⁶ is chosen from alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁷ is chosen from hydrogen, alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁸ is chosen from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl;

each R⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or when R⁸ and R⁹ are attached to adjacent atoms then R⁸ and R⁹ together with the atoms to which they are bonded form a cycloheteroalkyl or substituted cycloheteroalkyl ring; and

R¹¹ is chosen from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl;

with the provisos that:
when R¹ is [R⁵NH(CHR⁴)ₚC(O)]— then R² is —OR⁷; and
when R² is —[NR⁸(CHR⁹)_{q}C(O)OR⁷] then R¹ is not [R⁵NH(CHR⁴)ₚC(O)]—.

In certain embodiments, prodrugs of propofol may be chosen from any of the genuses or species of compounds of Formula (II) as disclosed in Gallop et al., U.S. Pat. No. 7,230,003:

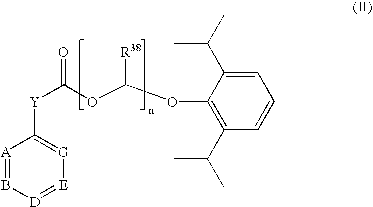

a pharmaceutically acceptable salt thereof, or a pharmaceutically acceptable solvate of any of the foregoing, wherein:

n is chosen from 0 and 1;

Y is chosen from a bond, CR²¹R²², NR²³, O, and S;

A is chosen from CR²⁴ and N;

B is chosen from CR²⁵ and N;

D is chosen from CR²⁶ and N;

E is chosen from CR²⁷ and N;

G is chosen from CR²⁸ and N;

R³⁸ is chosen from hydrogen, alkyl, substituted alkyl, alkoxycarbonyl, aryl, substituted aryl, arylalkyl, carbamoyl, substituted carbamoyl, cycloalkyl, substituted cycloalkyl, cycloheteroalkyl, heteroaryl, substituted heteroaryl, and heteroarylalkyl;

R²¹ and R²² are independently chosen from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl;

R²³ is chosen from hydrogen, alkyl, substituted alkyl, aryl, arylalkyl, cycloalkyl, and heteroaryl;

R²⁴ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR²⁹R³⁰)ᵣCO₂R³¹;

R²⁵ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR²⁹R³⁰)ᵣCO₂R³¹;

R²⁶ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR²⁹R³⁰)ᵣCO₂R³¹;

R²⁷ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR²⁹R³⁰)ᵣCO₂R³¹;

R²⁸ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]_{kZ}(CR²⁹R³⁰)ᵣCO₂R³¹;

W is chosen from a bond, —CR³²R³³, —NR³⁴, O, and S;

Z is chosen from —CR³⁵R³⁶, —NR³⁷, O, and S;

k is chosen from 0 and 1;

r is chosen from 1, 2, and 3;

each of R²⁹, R²⁹, R³¹, R³², R³³, R³⁵, and R³⁶ is independently chosen from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl; and

R³⁴ and R³⁷ are independently chosen from hydrogen, alkyl, substituted alkyl, aryl, arylalkyl, cycloalkyl, and heteroaryl;

with the provisos that:
at least one of A, B, D, E, and G is not N;
one and only one of R²⁴, R²⁵, R²⁶, R²⁷, or R²⁸ is —W[C(O)]ₖZ(CR²⁹R³⁰)ᵣCO₂R³¹; and
if k is 0 then W is a bond.

In certain embodiments, prodrugs of propofol may be chosen from any of the genuses or species of compounds of Formula (III) as disclosed in Xu et al., U.S. Application Publication No. 2006/0041011:

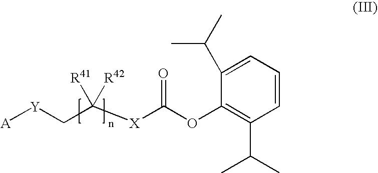

a pharmaceutically acceptable salt thereof, or a pharmaceutically acceptable solvate of any of the foregoing, wherein:

each R⁴¹ and R⁴² is independently chosen from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁴¹ and R⁴² together with the carbon atom to which they are bonded form a cycloalkyl, substituted cycloalkyl, cycloheteroalkyl, or substituted cycloheteroalkyl ring;

A is chosen from hydrogen, acyl, substituted acyl, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or A, Y, and one of R⁴¹ and R⁴² together with the atoms to which they are bonded form a cycloheteroalkyl or substituted cycloheteroalkyl ring;

Y is chosen from —O— and —NR⁴³—;

R⁴³ is chosen from hydrogen, alkyl, substituted alkyl, arylalkyl, and substituted arylalkyl;

n is an integer from 1 to 5;

X is chosen from —NR⁴⁴—, —O—, —CH₂, and —S—; and

R⁴⁴ is chosen from hydrogen, alkyl, substituted alkyl, arylalkyl, and substituted arylalkyl.

In certain embodiments, prodrugs of propofol may be chosen from any of the genuses or species of compounds of Formula (IV) as disclosed in Xu et al., U.S. patent application Ser. No. 11/180,064:

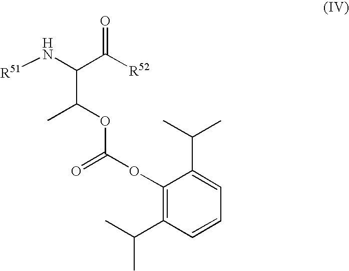

a pharmaceutically acceptable salt thereof, or a pharmaceutically acceptable solvate of any of the foregoing, wherein:

R⁵¹ is chosen from hydrogen, [R⁵⁵NH(CHR⁵⁴)ₚC(O)]—, R⁵⁶—, R⁵⁶C(O)—, and R⁵⁶OC(O)—;

R⁵² is chosen from —OR⁵⁷ and —[NR⁵⁸(CHR⁵⁹)_{q}C(O)OR⁵⁷];

p and q are independently chosen from 1 and 2;

each R⁵⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or when R⁵⁴ and R⁵⁵ are bonded to adjacent atoms then R⁵⁴ and R⁵⁵ together with the atoms to which they are bonded form a cycloheteroalkyl or substituted cycloheteroalkyl ring;

R⁵⁵ is chosen from hydrogen, R⁵⁶—, R⁵⁶C(O)—, and R⁵⁶OC(O)—;

R⁵⁶ is chosen from alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁵⁷ is chosen from hydrogen, alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁵⁸ is chosen from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl; and

each R⁵⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or when R⁵⁸ and R⁵⁹ are bonded to adjacent atoms then R⁵⁸ and R⁵⁹ together with the atoms to which they are bonded form a cycloheteroalkyl or substituted cycloheteroalkyl ring;

with the proviso that when R⁵² is —[NR⁵⁸(CHR⁵⁹)_{q}C(O)OR⁵⁷] then R⁵¹ is not [R⁵⁵NH(CHR⁵⁴)ₚC(O)]—.

In certain embodiments, a prodrug of propofol is 2-amino-3-methyl-3-(2,6-diisopropyl-phenoxycarbonyloxy)-propanoic acid (1):

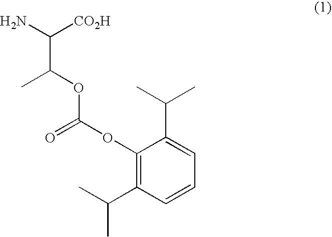

a pharmaceutically acceptable salt thereof, or a pharmaceutically acceptable solvate of any of the foregoing.

In certain embodiments of compound (1), the α-carbon of the amino acid residue is of the L-configuration. In certain embodiments of compound (1), the α-carbon of the amino acid residue is of the D-configuration.

In certain embodiments, a prodrug of propofol is 2-amino-3-(2,6-diisopropyl-phenoxycarbonyloxy)-propanoic acid (2) as disclosed in Xu et al., U.S. Application Publication No. 2006/0205969:

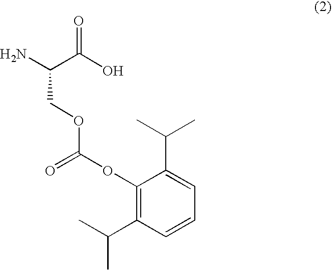

a pharmaceutically acceptable salt thereof, or a pharmaceutically acceptable solvate of any of the foregoing.

In certain embodiments, compound (2) may be a crystalline form of 2-amino-3-(2,6-diisopropyl-phenoxycarbonyloxy)-propanoic acid or pharmaceutically acceptable salts or solvates thereof. In certain embodiments, a prodrug of propofol of Formula (2) may be a crystalline form of (S)-2-amino-3-(2,6-diisopropylphenoxycarbonyloxy)-propanoic acid or pharmaceutically acceptable salts thereof, or pharmaceutically acceptable solvates thereof. In certain embodiments, a prodrug of propofol may be crystalline 2-amino-3-(2,6-diisopropylphenoxycarbonyloxy)-propanoic acid hydrochloride. In certain embodiments, a prodrug of propofol may be crystalline (S)-2-amino-3-(2,6-diisopropylphenoxycarbonyloxy)-propanoic acid hydrochloride. In certain embodiments, a prodrug of propofol may be crystalline (S)-2-amino-3-(2,6-diisopropylphenoxy-carbonyloxy)-propanoic acid hydrochloride having characteristic peaks (20) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 19.4°±0.2°, 20.1±0.2°, 21.3°±0.2°, 21.7°±0.2°, 22.5°±0.2°, 23.5°±0.2°, 24.4°±0.2°, 25.1°±0.2°, 26.8°±0.2°, 27.3°±0.2°, 27.8°±0.2°, 29.2°±0.2°±29.6°±0.2°, 30.4°±0.2°, and 33.4°±0.2° in an X-ray powder diffraction pattern. In certain embodiments, a prodrug of propofol may be crystalline (S)-2-amino-3-(2,6-diisopropylphenoxycarbonyloxy)-propanoic acid hydrochloride having characteristic peaks (20) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 20.1°±0.2°, 22.5°±0.2°, 23.5°±0.2°, 25.1°±0.2°, 29.2°±0.2°, 29.6°±0.2, and 33.4°±0.2° in an X-ray powder diffraction pattern.

In certain embodiments, a prodrug of propofol may be crystalline 2-amino-3-(2,6-diisopropylphenoxycarbonyloxy)-propanoic acid hydrochloride having a melting point from about 180° C. to about 200° C. In certain embodiments, a prodrug of propofol may be crystalline 2-amino-3-(2,6-diisopropylphenoxycarbonyloxy)-propanoic acid hydrochloride having a melting point from about 185° C. to about 195° C. In certain embodiments, a prodrug of propofol may be crystalline (S)-2-amino-3-(2,6-diisopropylphenoxycarbonyloxy)-propanoic acid hydrochloride having a melting point from about 188° C. to about 189° C.

In certain embodiments, a prodrug of propofol may be crystalline 2-amino-3-(2,6-diisopropylphenoxycarbonyloxy)-propanoic acid mesylate. In certain embodiments, a prodrug of propofol can be crystalline (S)-2-amino-3-(2,6-diisopropylphenoxycarbonyloxy)-propanoic acid mesylate. In certain embodiments, a prodrug of propofol may be crystalline (S)-2-amino-3-(2,6-diisopropylphenoxycarbonyloxy)-propanoic acid mesylate having characteristic peaks (2θ) at 4.2°±0.1°, 11.7°±0.1°, 12.1°±0.1°, 12.6°±0.1°, 16.8°±0.1°, 18.4°±0.2°, 21.0°±0.1°, 22.3°+0.1°, 22.8°+0.2°, 24.9°±10.2°, 25.3°+0.1°, 26.7°+0.2°, and 29.6°±0.1° in an X-ray powder diffraction pattern. In certain embodiments, a prodrug of propofol may be crystalline (s)-2-amino-3-(2,6-diisopropylphenoxycarbonyloxy)-propanoic acid mesylate having characteristic peaks (20) at 4.2°±0.1°, 12.6°±0.1°, 16.8°±0.1°, 21.0°+0.1°, 25.3°+0.1°, 2 and 29.6°±0.1° in an X-ray powder diffraction pattern.

In certain embodiments, a prodrug of propofol may be crystalline 2-amino-3-(2,6-diisopropylphenoxycarbonyloxy)-propanoic acid mesylate having a melting point from about 156° C. to about 176° C. In certain embodiments, a prodrug of propofol may be crystalline 2-amino-3-(2,6-diisopropylphenoxycarbonyloxy)-propanoic acid mesylate having a melting point from about 161° C. to about 172° C. In certain embodiments, a prodrug of propofol may be crystalline (S)-2-amino-3-(2,6-diisopropylphenoxycarbonyloxy)-propanoic acid mesylate having a melting point from about 166° C. to about 167° C.

Propofol prodrugs of Formulae (I)-(IV) may be administered orally and transported across cells (i.e., enterocytes) lining the lumen of the gastrointestinal tract. Certain of the compounds of structural Formulae (I)-(IV) may be substrates for the proton-coupled intestinal peptide transport system (PEPT1) (Leibach et al., Annu. Rev. Nutr. 1996, 16, 99-119), which mediates the cellular uptake of small intact peptides consisting of two or three amino acids that are derived from the digestion of dietary proteins. In the intestine, where small peptides are not effectively absorbed by passive diffusion, PEPT1 may act as a vehicle for the effective uptake of small peptides across the apical membrane of the gastric mucosa including propofol prodrugs of Formulae (I)-(IV).

Methods for determining whether propofol prodrugs of Formulae (I)-(IV) serve as substrates for the PEPT1 transporter are disclosed, for example, in Xu et al., U.S. patent application Ser. No. 11/180,064. In vitro systems using cells engineered to heterologously express the PEPT1 transport system or cell-lines that endogenously express the transporter (e.g. Caco-2 cells) may be used to assay transport of compounds of Formulae (I)-(IV) by the PEPT1 transporter. Standard methods for evaluating the enzymatic conversion of a propofol prodrug to propofol in vitro are disclosed, for example, in Xu et al., U.S. patent application Ser. No. 11/180,064.

Oral administration of propofol prodrugs to animals is described in Xu et al., U.S. Application Publication Nos. 2006/0041011 and 2006/0205969, and U.S. patent application Ser. No. 11/180,064, and illustrates that propofol prodrugs can afford significant enhancement in the oral bioavailability of propofol relative to the oral bioavailability of propofol when administered in an equivalent dosage form. In certain embodiments, a prodrug of propofol provides greater than 10% absolute oral bioavailability of propofol, i.e., compared to the bioavailability of propofol following intravenous administration of an equimolar dose of propofol itself. A prodrug of propofol that provides at least about 10 times higher oral bioavailability of propofol compared to the oral bioavailability of propofol itself, and in certain embodiments, at least about 40 times higher oral bioavailability of propofol compared to the oral bioavailability of propofol itself when orally administered in an equivalent dosage form (see, e.g., Xu et al., U.S. Application Publication Nos. 2006/0041011 and 2006/0205969, and U.S. patent application Ser. No. 11/180,064).

Methods of synthesizing prodrugs of propofol of Formula (I) are disclosed in Gallop et al., U.S. Pat. No. 7,220,875. Methods of synthesizing prodrugs of propofol of Formula (II) are disclosed in Gallop et al., U.S. Pat. No. 7,230,003. Methods of synthesizing prodrugs of propofol of Formulae (III) are disclosed in Xu et al., U.S. Application Publication No. 2006/0041011. Methods of synthesizing prodrugs of propofol of Formulae (IV) are disclosed in Xu et al., U.S. patent application Ser. No. 11/180,064. Methods of synthesizing and crystallizing prodrugs of propofol of Formula (2) are disclosed in Xu et al., U.S. Application Publication No. 2006/0205969.

Propofol prodrugs of Formulae (I)-(IV) are distinguished from other propofol prodrugs by their ability to provide high oral bioavailability of propofol. Various prodrugs of propofol have been developed that enhance the aqueous solubility of propofol for intravenous administration (Stella et al., U.S. Pat. Nos. 6,204,257 and 6,872,838; Hendler et al., U.S. Pat. Nos. 6,254,853 and 6,362,234; Jenkins et al., U.S. Pat. No. 6,815,555; Wingard et al., U.S. Application Publication No. 2005/0203068; Marappan et al., U.S. Pat. No. 7,250,412; Orlando et al., U.S. Application Publication No. 2005/0267169; Fechner et al., Anesthesiology 2003, 99, 303-313; Fechner et al., Anesthesiology 2004, 101, 626-639; Struys et al., Anesthesiology 2005, 103, 730-43; and Gibiansky et al., Anesthesiology 2005, 103, 718-729). While the use of such prodrugs for oral administration is disclosed, there is no evidence to suggest that any of the propofol prodrugs intended for use in aqueous intravenous formulations provides clinically relevant systemic propofol concentrations when orally administered.

Any of the forms of propofol disclosed herein may exhibit sufficient stability to enzymatic and/or chemical degradation in the gastrointestinal tract resulting in enhanced oral bioavailability of the form of propofol and/or propofol metabolite. The forms of propofol may also exhibit enhanced passive and/or active gastrointestinal absorption compared to propofol. In certain embodiments, a form of propofol is chosen from a propofol prodrug and a propofol tight-ion pair complex. In certain embodiments, a form of propofol is a propofol prodrug and is chosen from a compound of Formula (I) to Formula (IV). In certain embodiments, a form of propofol is compound (2), and in certain embodiments, is (S)-2-amino-3-(2,6-diisopropylphenoxycarbonyloxy)-propanoic acid.

### Pharmaceutical Compositions

Forms of propofol providedbythepresent disclosure may be formulated into pharmaceutical compositions for use in oral dosage forms to be administered to patients.

Pharmaceutical compositions comprise at least one form of propofol and at least one pharmaceutically acceptable vehicle. A pharmaceutical composition can comprise a therapeutically effective amount of at least one form of propofol and at least one pharmaceutically acceptable vehicle. Pharmaceutically acceptable vehicles include diluents, adjuvants, excipients, and carriers. Pharmaceutical compositions can be produced using standard procedures (see, e.g., Remington's The Science and Practice of Pharmacy, 21st edition, Lippincott, Williams & Wilcox, 2005). Pharmaceutical compositions may take any form appropriate for oral delivery such as solutions, suspensions, emulsions, tablets, pills, pellets, granules, capsules, capsules containing liquids, powders, and the like. Pharmaceutical compositions of the present disclosure may be formulated so as to provide immediate, sustained, or delayed release of a form of propofol after administration to the patient by employing procedures known in the art (see, e.g., Allen et al., “Ansel's Pharmaceutical Dosage Forms and Drug Delivery Systems,” 8th edition, Lippincott, Williams & Wilkins, August 2004).

Pharmaceutical compositions may include an adjuvant that facilitates absorption of a form of propofol through the gastrointestinal epithelia. Such enhancers may, for example, open the tight-junctions in the gastrointestinal tract or modify the effect of cellular components, such as p-glycoprotein and the like. Suitable enhancers include alkali metal salts of salicylic acid, such as sodium salicylate, caprylic, or capric acid, such as sodium caprylate or sodium caprate, sodium deoxycholate, and the like. P-glycoprotein modulators are described in Fukazawa et al., U.S. Pat. No. 5,112,817 and Pfister et al., U.S. Pat. No. 5,643,909. Absorption enhancing compounds and materials are described in Burnside et al., U.S. Pat. No. 5,824,638, and Meezam et al., U.S. Application Publication No. 2006/0046962. Other adjuvants that enhance permeability of cellular membranes include resorcinol, surfactants, polyethylene glycol, and bile acids. Adjuvants may also reduce enzymatic degradation of a compound of a form of propofol. Microencapsulation using protenoid microspheres, liposomes, or polysaccharides may also be effective in reducing enzymatic degradation of administered compounds.

Forms of propofol provided by the present disclosure may be formulated in unit oral dosage forms. Unit oral dosage forms refer to physically discrete units suitable for dosing to a patient undergoing treatment, with each unit containing a predetermined quantity of a form of propofol. Oral dosage forms comprising at least one form of propofol are administered to patients as a dose, with each dose comprising one or more oral dosage forms. A dose may be administered once a day, twice a day, or more than twice a day, such as three or four times per day. A dose can be administered at a single point in time or during a time interval. Oral dosage forms comprising a form of propofol may be administered alone or in combination with other drugs for treating the same or different disease, and may continue as long as required for effective treatment of the disease. Oral dosage forms comprising form of propofol may provide a concentration of propofol in the plasma, blood, or tissue of a patient over time, following oral administration of the dosage form to the patient. The propofol concentration profile may exhibit an AUC that is proportional to the dose of the form of propofol.

A dose comprises an amount of a form of propofol calculated to produce an intended therapeutic effect. An appropriate amount of a form of propofol to produce an intended therapeutic effect will depend, in part, on the oral bioavailability of propofol provided by the form of propofol, by the pharmacokinetics of the form of propofol, and by the properties of the dosage form used to administer the form of propofol. A therapeutically effective dose of a form of propofol may comprise from about 10 mg-equivalents to about 5,000 mg-equivalents of propofol, from about 50 mg-equivalents to about 2,000 mg-equivalents of propofol, and in certain embodiments, from about 100 mg-equivalents to about 1,000 mg-equivalents of propofol. In certain embodiments, a therapeutically effective dose of a form of propofol provides a blood concentration of propofol from about 10 ng/mL to about 5,000 ng/mL, in certain embodiments from about 100 ng/mL to about 2,000 ng/mL, and in certain embodiments from about 200 ng/mL to about 1,000 ng/mL for a continuous period of time following oral administration of a dosage form comprising a form of propofol to a patient. In certain embodiments, a therapeutically effective dose of a form of propofol provides a blood concentration of propofol that is therapeutically effective for treating a disease in a patient, and that is less than a concentration effective in causing sedation in the patient, for example, less than about 1,500 ng/mL or less than about 2,000 ng/mL. In certain embodiments, a therapeutically effective dose of a form of propofol provides a blood concentration of propofol that is therapeutically effective and that is less than a concentration effective for the maintenance of general anesthesia (e.g., a sub-hypnotic concentration), for example, less than about 3,000 ng/mL or less than about 10,000 ng/mL.

Oral dosage forms comprising a form of propofol may have immediate release or controlled release characteristics. Immediate release oral dosage forms release the form of propofol from the dosage form within about 30 minutes following ingestion. In certain embodiments, an oral dosage form provided by the present disclosure may be a controlled release dosage form. Controlled delivery technologies may improve the absorption of a drug in a particular region or regions of the gastrointestinal tract. Controlled drug delivery systems may be designed to deliver a drug in such a way that the drug level is maintained within a therapeutically effective blood concentration range for a period as long as the system continues to deliver the drug at a particular rate. Controlled drug delivery may produce substantially constant blood levels of a drug as compared to fluctuations observed with immediate release dosage forms. For some diseases maintaining a controlled concentration of propofol in the blood or in a tissue throughout the course of therapy is desirable. Immediate release dosage forms may cause blood levels to peak above the level required to elicit the desired response, which may cause or exacerbate side effects. Controlled drug delivery may result in optimum therapy, reduce the frequency of dosing, and reduce the occurrence, frequency, and/or severity of side effects. Examples of controlled release dosage forms include dissolution controlled systems, diffusion controlled systems, ion exchange resins, osmotically controlled systems, erodable matrix systems, pH independent formulations, gastric retention systems, and the like.

The appropriate oral dosage form for a particular form of propofol may depend, at least in part, on the gastrointestinal absorption properties of the form of propofol, the stability of the form of propofol in the gastrointestinal tract, the pharmacokinetics of the form of propofol, and the intended therapeutic profile of propofol. An appropriate controlled release oral dosage form may be selected for a particular form of propofol. For example, gastric retention oral dosage forms may be appropriate for forms of propofol absorbed primarily from the upper gastrointestinal tract, and sustained release oral dosage forms may be appropriate for forms of propofol absorbed primarily form the lower gastrointestinal tract.

Gastric retention dosage forms, i.e., dosage forms designed to be retained in the stomach for a prolonged period of time, can increase the bioavailability of drugs that are most readily absorbed from the upper gastrointestinal tract. The residence time of a conventional dosage form in the stomach is 1 to 3 hours. After transiting the stomach, there is approximately a 3 to 5 hour window of bioavailability before the dosage form reaches the colon. However, if the dosage form is retained in the stomach, the drug can be released before it reaches the small intestine and will enter the intestine in solution in a state in which it can be more readily absorbed. Another use of gastric retention dosage forms is to improve the bioavailability of a drug that is unstable to the basic conditions of the intestine (see, e.g., Hwang et al., Critical Reviews in Therapeutic Drug Carrier Systems, 1998, 15, 243-284). To enhance drug absorption from the upper gastrointestinal tract, several gastric retention dosage forms have been developed. Examples include, hydrogels (see, e.g., Gutierrez-Rocca et al., U.S. Application Publication No. 2003/0008007), buoyant matrices (see, e.g., Lohray et al., Application Publication No. 2006/0013876), polymer sheets (see, e.g., Mohammad, Application Publication No. 2005/0249798), microcellular foams (see, e.g., Clarke et al., Application Publication No. 2005/0202090), and swellable dosage forms (see, e.g., Edgren et al., U.S. Application Publication No. 2005/0019409; Edgren et al., U.S. Pat. No. 6,797,283; Jacob et al., U.S. Application Publication No. 2006/0045865; Ayres, U.S. Application Publication No. 2004/0219186; Gusler et al., U.S. Pat. No. 6,723,340; Flashner-Barak et al., U.S. Pat. No. 6,476,006; Wong et al., U.S. Pat. Nos. 6,120,803 and 6,548,083; Shell et al., U.S. Pat. No. 6,635,280; and Conte et al., U.S. Pat. No. 5,780,057).

In swelling and expanding systems, dosage forms that swell and change density in relation to the surrounding gastric content may be retained in the stomach for longer than conventional dosage forms. Dosage forms can absorb water and swell to form a gelatinous outside surface and float on the surface of gastric content surface while maintaining integrity before releasing a drug. Fatty materials may be added to impede wetting and enhance flotation when hydration and swelling alone are insufficient. Materials that release gases may also be incorporated to reduce the density of a gastric retention dosage form. Swelling also may significantly increase the size of a dosage form and thereby impede discharge of the non-disintegrated swollen solid dosage form through the pylorus into the small intestine. Swellable dosage forms may be formed by encapsulating a core containing drug and a swelling agent, or by combining a drug, swelling agent, and one or more erodible polymers.

Gastric retention dosage forms may also be in the form of folded thin sheets containing a drug and water-insoluble diffusible polymer that opens in the stomach to its original size and shape so as to be sufficiently large to prevent or inhibit passage of the expanded dosage form through the pyloric sphincter.

Floating and buoyancy gastric retention dosage forms are designed to trap gases within sealed encapsulated cores that can float on the gastric contents, and thereby be retained in the stomach for a longer time, e.g., 9 to 12 hours. Due to the buoyancy effect, these systems provide a protective layer preventing the reflux of gastric content into the esophageal region and may also be used for controlled release devices. A floating system may, for example, contain hollow cores containing drug coated with a protective membrane. The trapped air in the cores floats the dosage form on the gastric content until the soluble ingredients are released and the system collapses. In other floating systems, cores comprise drug and chemical substances capable of generating gases when activated. For example, coated cores, comprising carbonate and/or bicarbonate generate carbon dioxide in the reaction with hydrochloric acid in the stomach or incorporated organic acid in the system. The gas generated by the reaction is retained to float the dosage form. The inflated dosage form later collapses and clears from the stomach when the generated gas permeates slowly through the protective coating.

Bioadhesive polymers may also provide vehicles for controlled delivery of drugs to a number of mucosal surfaces in addition to the gastric mucosa (see, e.g., Mathiowitz et al., U.S. Pat. No. 6,235,313; and Illum et al., U.S. Pat. No. 6,207,197). Bioadhesive systems can be designed by incorporation of a drug and other excipients within a bioadhesive polymer. On ingestion, the polymer hydrates and adheres to the mucus membrane of the gastrointestinal tract. Bioadhesive polymers may be selected that adhere to a desired region or regions of the gastrointestinal tract. Bioadhesive polymers may be selected to optimized delivery to targeted regions of the gastrointestinal tract including the stomach and small intestine. The mechanism of the adhesion is thought to be through the formation of electrostatic and hydrogen bonding at the polymer-mucus boundary. Jacob et al., U.S. Application Publication Nos. 2006/0045865 and 2005/0064027 disclose bioadhesive delivery systems useful for drug delivery to both the upper and lower gastrointestinal tract.

Ion exchange resins have been shown to prolong gastric retention, potentially by adhesion.

Gastric retention oral dosage forms may be used for delivery of drugs that are absorbed mainly from the upper gastrointestinal tract. For example, certain forms of propofol may exhibit limited colonic absorption, and be absorbed primarily from the upper gastrointestinal tract. Thus, dosage forms that release the form of propofol in the upper gastrointestinal tract and/or retard transit of the dosage form through the upper gastrointestinal tract will tend to enhance the oral bioavailability of the form of propofol or propofol metabolite.

Polymer matrices have also been used to achieve controlled release of drug over a prolonged period of time. Sustained or controlled release may be achieved by limiting the rate by which the surrounding gastric fluid can diffuse through the matrix and reach the drug, dissolve the drug and diffuse out again with the dissolved drug, or by using a matrix that slowly erodes, continuously exposing fresh drug to the surrounding fluid. Disclosures of polymer matrices that function by these methods are found, for example, in Skinner, U.S. Pat. Nos. 6,210,710 and 6,217,903; Rencher et al., U.S. Pat. No. 5,451,409; Kim, U.S. Pat. No. 5,945,125; Kim, PCT International Publication No. WO 96/26718; Ayer et al., U.S. Pat. No. 4,915,952; Akhtar et al., U.S. Pat. No. 5,328,942; Fassihi et al., U.S. Pat. No. 5,783,212; Wong et al., U.S. Pat. No. 6,120,803; and Pillay et al., U.S. Pat. No. 6,090,411.

Other drug delivery devices that remain in the stomach for extended periods of time include, for example, hydrogel reservoirs containing particles (Edgren et al., U.S. Pat. No. 4,871,548); swellable hydroxypropylmethylcellulose polymers (Edgren et al., U.S. Pat. No. 4,871,548); planar bioerodible polymers (Caldwell et al., U.S. Pat. No. 4,767,627); polymers comprising a plurality of compressible retention arms (Curatolo et al., U.S. Pat. No. 5,443,843); hydrophilic water-swellable, cross-linked polymer particles (Shell, U.S. Pat. No. 5,007,790); and albumin-cross-linked polyvinylpyrrolidone hydrogels (Park et al., J. Controlled Release 1992, 19, 131-134).

In certain embodiments, forms of propofol may be practiced with a number of different dosage forms adapted to provide sustained release of the form of propofol upon oral administration. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's Pharmaceutical Sciences,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2). Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's: The Science and Practice of Pharmacy,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2).

In diffusion-controlled systems, water-insoluble polymers control the flow of fluid and the subsequent egress of dissolved drug from the dosage form. Both diffusional and dissolution processes are involved in release of drug from the dosage form. In reservoir devices, a core comprising a drug is coated with the polymer, and in matrix systems, the drug is dispersed throughout the matrix. Cellulose polymers such as ethylcellulose or cellulose acetate can be used in reservoir devices. Examples of materials useful in matrix systems include methacrylates, acrylates, polyethylene, acrylic acid copolymers, polyvinylchloride, high molecular weight polyvinylalcohols, cellulose derivates, and fatty compounds such as fatty acids, glycerides, and carnauba wax.

In dissolution-controlled systems, the rate of dissolution of a drug is controlled by slowly soluble polymers or by microencapsulation. Once the coating is dissolved, the drug becomes available for dissolution. By varying the thickness and/or the composition of the coating or coatings, the rate of drug release can be controlled. In some dissolution-controlled systems, a fraction of the total dose may comprise an immediate-release component. Dissolution-controlled systems include encapsulated/reservoir dissolution systems and matrix dissolution systems. Encapsulated dissolution systems may be prepared by coating particles or granules of drug with slowly soluble polymers of different thickness or by microencapsulation. Examples of coating materials useful in dissolution-controlled systems include gelatin, carnauba wax, shellac, cellulose acetate phthalate, and cellulose acetate butyrate. Matrix dissolution devices may be prepared, for example, by compressing a drug with a slowly soluble polymer carrier into a tablet form.

The rate of release of drug from osmotic pump systems is determined by the inflow of fluid across a semipermeable membrane into a reservoir, which contains an osmotic agent. The drug is either mixed with the agent or is located in a reservoir. The dosage form contains one or more small orifices from which dissolved drug is pumped at a rate determined by the rate of entrance of water due to osmotic pressure. As osmotic pressure within the dosage form increases, the drug is released through the orifice(s). The rate of release is constant and may be controlled within limits yielding relatively constant blood concentrations of the drug. Osmotic pump systems may provide a constant release of drug independent of the environment of the gastrointestinal tract. The rate of drug release may be modified by altering the osmotic agent and the size of the one or more orifices.

Release of drug from erosion-controlled systems is determined by the erosion rate of a carrier polymer matrix. Drug is dispersed throughout the polymer matrix and the rate of drug release depends on the erosion rate of the polymer. The drug-containing polymer may degrade from the bulk and/or from the surface of the dosage form.

Sustained release oral dosage forms may be in any appropriate form suitable for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, and the like.

Sustained release oral dosage forms may release a form of propofol from the dosage form to facilitate the ability of the form of propofol or propofol metabolite to be absorbed from an appropriate region of the gastrointestinal tract, for example, in the small intestine, or in the colon. In certain embodiments, sustained release oral dosage forms may release a form of propofol from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms may release a form of propofol from the dosage form in a delivery pattern in which from about 0 wt % to about 20 wt % of the form of propofol is released in about 0 to about 4 hours, about 20 wt % to about 50 wt % of the form of propofol is released in about 0 to about 8 hours, about 55 wt % to about 85 wt % of the form of propofol is released in about 0 to about 14 hours, and about 80 wt % to about 100 wt % of the form of propofol is released in about 0 to about 24 hours. In certain embodiments, sustained release oral dosage forms may release a form of propofol from the dosage form in a delivery pattern in which from about 0 wt % to about 20 wt % of the form of propofol is released in about 0 to about 4 hours, about 20 wt % to about 50 wt % of the form of propofol is released in about 0 to about 8 hours, about 55 wt % to about 85 wt % of the form of propofol is released in about 0 to about 14 hours, and about 80 wt % to about 100 wt % of the form of propofol is released in about 0 to about 20 hours. In certain embodiments, sustained release oral dosage forms may release a form of propofol from the dosage form in a delivery pattern in which from about 0 wt % to about 20 wt % of the form of propofol is released in about 0 to about 2 hours, about 20 wt % to about 50 wt % of the form of propofol is released in about 0 to about 4 hours, about 55 wt % to about 85 wt % of the form of propofol is released in about 0 to about 7 hours, and about 80 wt % to about 100 wt % of the form of propofol is released in about 0 to about 8 hours.

Regardless of the specific form of oral dosage form used, a form of propofol may be released from the orally administered dosage form over a sufficient period of time to provide prolonged therapeutic concentrations of propofol in blood of a patient. Following oral administration, dosage forms comprising a form of propofol may provide a therapeutically effective concentration of propofol in the blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous period of time during which a therapeutically effective blood concentration of propofol is maintained may begin shortly after oral administration or following a time interval.

In certain embodiments, it may be desirable that the blood concentration of propofol be maintained at a level between a concentration that causes moderate sedation in the patient and a minimum therapeutically effective concentration for treating a disease associated with oxidative stress for a continuous period of time. The blood concentration of propofol that causes moderate sedation (or anesthesia) in a patient can vary depending on the individual patient. Generally, a blood propofol concentration from about 1,500 ng/mL to about 2,000 ng/mL will produce moderate sedation, while a blood propofol concentration from about 3,000 ng/mL to about 10,000 ng/mL is sufficient to maintain general anesthesia. In certain embodiments, a minimum therapeutically effective blood propofol concentration will be about 10 ng/mL, about 20 ng/mL, about 50 ng/mL, about 100 ng/mL, about 100 ng/mL, about 200 ng/mL, about 400 ng/mL, or about 600 ng/mL. In certain embodiments, a therapeutically effective blood concentration of propofol for treating a disease associated with oxidative stress is from about 10 ng/mL to less than about 5,000 ng/mL. In certain embodiments, a therapeutically effective blood concentration of propofol for treating a disease associated with oxidative stress is from about 10 ng/mL to less than a sedative concentration. In certain embodiments, a therapeutically effective blood concentration of propofol for treating a disease associated with oxidative stress is from about 200 ng/mL to about 1,000 ng/mL. In certain embodiments, methods of the present disclosure provide a blood propofol concentration that, following oral administration to a patient, does not produce sedation and/or anesthesia in the patient.

A therapeutically effective propofol blood concentration for treating a disease associated with oxidative stress in a patient can also be defined in terms of the plasma concentration or pharmacokinetic profile. Thus, in certain embodiments, following oral administration of a dosage form comprising a form of propofol to a patient, the maximum propofol blood concentration, Cₘₐₓ, is less than that which causes moderate sedation, for example, is less than about 1,500 ng/mL to about 2,000 ng/mL. In certain embodiments, following oral administration of a dosage form comprising a form of propofol to a patient, the propofol blood AUC during a 4-hour period may range from about 800 ng·h/mL to about 3,200 ng·h/mL and not cause sedation at any time following oral administration. In certain embodiments, following oral administration of a dosage form comprising a form of propofol to a patient, the propofol blood AUC during an 8-hour period may range from about 1,600 ng·h/mL to about 6,400 ng·h/mL and not cause sedation at any time following oral administration. In certain embodiments, following oral administration of a dosage form comprising a form of propofol to a patient, the propofol blood AUC during a 12-hour period may range from about 2,400 ng·h/mL to about 9,200 ng·h/mL and not cause sedation at any time following oral administration. In certain embodiments, following oral administration of a dosage form comprising a form of propofol to a patient, the propofol blood AUC during a 16-hour period may range from about 3,200 ng·h/mL to about 12,800 ng·h/mL and not cause sedation at any time following oral administration. In certain embodiments, following oral administration of a dosage form comprising a form of propofol to a patient, the propofol blood AUC during a 32-hour period may range from about 4,000 ng·h/mL to about 16,000 ng·h/mL and not cause sedation at any time following oral administration.

In certain embodiments, a form of propofol may be absorbed from the gastrointestinal tract and enter the systemic circulation intact. In certain embodiments, a form of propofol exhibits an oral bioavailability of the form of propofol greater than about 40% that of an equivalent intravenous dose of the form of highly orally bioavailable propofol, greater than about 60%, and in certain embodiments greater than about 80%. In certain of the foregoing embodiments, a form of propofol exhibits an oral bioavailability of propofol greater than about 10% that of an equivalent intravenous dose of propofol, greater than about 20%, greater than about 40% and in certain embodiments greater than about 60%.

### Methods of Use

Forms of propofol that provide a high oral bioavailability of propofol and dosage forms comprising such forms of propofol may be used to treat diseases associated with oxidative stress. Methods provided by the present disclosure comprise treating a disease associated with oxidative stress in a patient by administering to a patient in need of such treatment a therapeutically effective amount of at least one form of propofol that provides a high oral bioavailability of propofol. Diseases associated with oxidative stress include metabolic diseases, cardiovascular diseases, neurological diseases, liver diseases, and pulmonary diseases.

### Metabolic Diseases

Metabolic diseases include prediabetes, diabetes mellitus type I, diabetes mellitus type II, metabolic syndrome, hypertension, obesity, and dyslipidemia.

The forms of diabetes mellitus are characterized by chronic hyperglycemia and the development of diabetes-specific microvascular pathology, generally associated with accelerated atherosclerotic macrovascular disease affecting arteries that supply the heart, brain, and lower extremities (see e.g., Brownlee, Diabetes 2005, 54 (June), 1615-1625; and Brownlee, Nature 2001, 414(13 December), 813-820). Diabetes selectively damages cells, such as endothelial cells, in which the glucose transport rate does not decline rapidly as a result of hyperglycemia, leading to high intracellular glucose concentrations. The microvascular and macrovascular pathologies resulting from hyperglycaemia are believed to result from increased polyol pathway flux, increased advanced glycation end-product (AGE) formation, activation of protein kinase C(PKC) isoforms, and increased hexosamine pathway flux. These pathogenic mechanisms, in turn, are a consequence of hyperglycaemia-induced oxidative stress characterized by an increased level of intracellular ROS such as the overproduction of superoxide in the mitochondrial electron-transport chain as well as by a decrease in enzymatic and non-enzymatic antioxidant defenses (Brownlee, Id.; Hammes, J Diabetes and Its Complications 2003, 17, 16-19; Nishikawa et al., Nature 2000, 404 (13 April), 787-790; Bonnefont-Rousselot, Cur Opinion Clin Nutrition Metabolic Care 2002, 5, 561-568; Johansen et al., Cardiovascular Diabetology 2005, 4(1), 5; and Houstis et al., Nature 2006, 440 (13 April), 944-948).

Independent of these mechanisms, excess superoxide also directly inhibits the activity of the anti-atherogenic enzymes endothelial nitric oxide synthase (eNOS) and prostacyclin synthase (Santilli et al., Horm Metab Res 2004, 36, 319-335). Hyperglycemia-induced reactive oxygen overproduction reduces eNOS activity in diabetic aortas by 65% and prostacyclin synthase activity by 95%. Endothelium-derived nitric oxide (NO) is a potent chemical mediator with antiatherogenic properties, such as stimulation of vasorelaxation and repression of endothelial leukocyte adhesion molecules, platelet aggregation, and smooth muscle cell proliferation (Forstermann et al., Hypertension 1994, 23, 1121-1131; Joannides et al., Circulation 1995, 92, 1314-1319; Moncada and Higgs, New Eng J Med 1993, 329, 2002-2012; Hink et al., Circ Res 2001, 88, 14-22; Bitar et al., Eur J Pharmacology 2005, 511, 53-64; and Dandona and Chaudhuri, Med Clin N Am 2004, 88, 911-931). Endothelial dysfunction contributes significantly to diabetic vascular disease and is an important factor in the development of diabetic neuropathy. Some of the mechanisms attributed to diabetes induced endothelium dysfunction include impaired signal transduction pathways or substrate availability, impaired release or increased metabolism of vasodilatory mediators, increased release of vascular constricting factors, and decreased reactivity of the smooth muscle to vasodilatory mediators.

Efforts to interrupt the overproduction of superoxide by the mitochondrial electron-transport chain and thereby normalize polyol pathway flux, AGE formation, PKC activation, hexosamine pathway flux and NF-κB activation using conventional antioxidants such as reactive oxygen scavengers have not been conclusive (Kowluru et al., Diabetes 2001, 50, 1938-1942; Ting et al., J Clin Invest 1996, 97, 22-28; and Lancet 2000, 355, 253-259). Clinical trials investigating the effect of the antioxidant vitamin E (α-tocopherol) have also failed to conclusively demonstrate benefits on cardiovascular complications associated with diabetes (Giugliano et al., Diabetes Care 1996, 19(3), 257-267; Ceriello, Diabetes Care 2003, 26(5), 1589-1596; and Ceriello and Motz, Arterioscler Thromb Vasc Biol 2004, 24(5), 816-823).

Studies do suggest, however, that intracellular ROS scavengers may be effective in addressing diabetic complications. For example, many of the drugs used in the pharmacotherapy in diabetes including thiazolidinediones, HMG-CoA reductase inhibitors (statins), ACE inhibitors, AT-1 blockers, calcium channel blockers and inhibitors of the rennin-angiotensin system have been shown to have intracellular antioxidant activity in addition to their primary pharmacological actions (Ceriello, Diabetes Care 2003, 26(5), 1589-1596). In addition to its glucose-lowering effects, the antidiabetic sulfonylurea, gliclazide ameliorates impaired vasoregulation in diabetic patients by acting as intracellular ROS scavengers (Mamputo and Renier, J Diabetes and Its Complications 2002, 16, 284-293; and Fava et al., Diabetic Medicine, 2002, 19, 752-757). Troglitazone, a thiazolidinedione drug used to treat diabetes by enhancing insulin sensitivity through its function as a ligand for peroxisome proliferator-activated receptor γ (PPAR-γ) has also been shown to have antioxidant properties, which may contribute to its efficacy (Petersen et al., Diabetes, 2000, 49, 827-831; Loefsky, J Clin Investigation 2000, 106, 467-472; and Touyz and Schiffrin, Vascular Pharmacology 2006, 45, 19-28). Troglitazone also has vasodilating and blood pressure-lowering effects, which may be mediated by increased eNOS protein expression and antioxidant activity (Goya et al., J Diabetes and Its Complications 2006, 20, 3365-342). Other antidiabetic thiazolidinedione drugs such as pioglitazone lack such antioxidant activity (Inoue et al., Biochemical and Biophysical Res Communications 1997, 235, 113-116; and Maritim et al., J Biochem Molecular Toxicology 2003, 17(1), 24-38). Furthermore, conventional antioxidants such as α-tocopherol have been shown to increase eNOS protein expression (Rodriquez et al., Atherosclerosis, 2002, 165, 33-40; and Newaz et al., Hypertension 1999, 12, 839-844).

Other conditions associated with diabetes such as metabolic syndrome, dyslipidemia, obesity, and hypertension are also associated with oxidative stress and may therefore benefit from improved antioxidant therapies (Moller and Kaufman, Annu Rev Med 2005, 56, 45-62; and Cifuentes and Pagano, Curr Opin Nephrol Hypertens 2006, 15(2), 179-86)). Metabolic syndrome refers to a cluster of interrelated common clinical disorders, including obesity, insulin resistance (Diabetes Mellitus Type II), glucose intolerance, hypertension, and dyslipidemia (hypertriglyceridemia and low HDL cholesterol levels). Dyslipidemias include lipoprotein overproduction or deficiency. Hypertension, or high blood pressure, is defined as a repeatedly elevated blood pressure exceeding 140 over 90 mm-Hg and a systolic pressure above 140 mm-Hg with a diastolic pressure above 90 mm-Hg.

The efficacy of compounds provided by the present disclosure for treating metabolic diseases can be assessed using animal models and in clinical trials. For example, animal models of diabetes are disclosed in Rees and Alcolado, Diabetic Medicine 2005, 22, 359-370; and Shafrir et al., eds, “Animal Models of Diabetes,” CRC Press, Ed. 2, 2007.

### Cardiovascular Diseases

Cardiovascular diseases and disorders include atherosclerosis, arteriosclerosis, hyperlipidemia, ischemia-reperfusion injury, stenosis, ischemia, angina, myocardial infarction, peripheral artery disease, hypertension, arterial aneurysms, cardiomegaly, tachycardia/bradycardia/arrhythmia, cardiac arrest, cardiomyopathy, congestive heart failure, and stroke.

Oxidative stress is implicated in the pathogenesis of cardiovascular disease (Kevin, Anesth Analg 2005, 101, 1275-87; and Molavi and Mehta, Curr Opin Cardiol 2004, 19(5), 488-493). For example, the impairment of endothelial NO production has been suggested to cause cardiovascular diseases (Dusting, Exs 1996, 76, 33-55), and in the pathogenesis of atherosclerosis is endothelial cell dysfunction (Lusis, Nature 2000, 407, 233-242). Sufficient constitutive NO production in endothelium is important not only for fine tuning of vascular tone but also for the prevention of the development of thrombosis and coagulation. In hyperlipidemia and atherosclerosis eNOS becomes dysfunctional and produces superoxide rather than NO (Kawashima and Yokoyama, Arterioscler Thromb Vasc Biol 2004, 24, 998-1005). Oxidative stress is also believed to play a role in the pathogenesis of stroke and congestive heart failure (see e.g., Mariani et al., J. Chromatogr. B. 2005, 827, 65-67). Free radicals and their nonradical reactants are recognized as critical mediators of cardiac injury during ischemia and reperfusion. They have been implicated in reversible postischemic contractile dysfunction, cardiac cell death, dysrhythmias, and in chronic cardiovascular disease.

Administration of exogenous antioxidants has been investigated to treat chronic cardiovascular disease. Propofol has been shown to be protective in experimental models of injury to organs including the brain, liver, and heart. The cardioprotective effects of propofol are believed to result form preservation of endothelium-dependent vasodilation, which is impaired by oxidative stress (Young et al., Eur J Anaesthesiol 1997, 14, 320-26; and Navapurkar et al., Anesth Analg 1998, 87, 1152-57). The vasodilator activity of propofol is not necessarily mediated or modulated by the release of nitric oxide, (Kaye et al., Acta Anaesthesiol Scand 1999, 43(4), 431-7), and may be the result of a number of mechanisms including activation of the BK(Ca) K⁺ channel (a high conductance Ca₂⁺ sensitive K⁺ channel) (Kockgether-Radke et al., Eur J. Anaesthesiol 2004, 21(3), 226-30). In heart models, propofol is protective against peroxidative damage and functional impairment induced by exogenous H₂O₂ (Kokita and Hara, Anesthesiology 1996, 84, 117-27) and by ischemia-reperfusion (Kokita et al., Anesth Analg 1998, 86, 252-258). Propofol also has been shown to exhibit cardioprotective properties (Kato and Foex, Can J Anesth 2002, 49(8), 777-791), possible by activating protein kinase C(PKC) in cardiomyocytes (Wickley et al., Anesthesiology 2006, 104, 70-7). It has been suggested that propofol-induced cardioprotection may partly result form a direct effect on myocardial calcium influx, or from inhibition of mitochondrial permeability transition. (Kevin et al., Anesth Analg 2005, 101, 1275-87).

Antioxidants such as propofol may also exert a therapeutic effect by inhibiting free fatty acid (FFA) oxidation. Energy metabolism in the heart can be manipulated indirectly as well as by the use of agents that directly act on the heart to shift energy substrate use away from fatty acid metabolism and toward glucose metabolism, which is more efficient in terms of ATP production per mole of oxygen used. One way to increase glucose oxidation and to decrease fatty acid metabolism in the heart is to decrease circulating fatty acid levels. This can be achieved by the administration of glucose-insulin solutions, nicotinic acid, and β-adrenergic blocking drugs. Another approach involves directly modifying substrate use by the heart. Pharmacological agents that inhibit fatty acid oxidation include beta-oxidation inhibitors, the so-called 3-ketoacyl-coenzyme A thiolase inhibitors, such as trimetazidine and ranolazine. Inhibition of oxidative phosphorylation and fatty acid substrates has been shown to shift substrate use from fatty acid to glucose.

An important metabolic alteration in patients with diabetes is the increase in FFA concentrations and the increased skeletal muscle and myocardial FFA uptake and oxidation. The increased uptake and utilization of FFA and the reduced utilization of glucose as a source of energy during stress and ischemia contribute to hyperglycemia in patients with non-insulin dependent diabetes mellitus and to the increased susceptibility of diabetic hearts to myocardial ischemia and to a greater decrease of myocardial performance for a given amount of ischemia compared with nondiabetic hearts.

Trimetazidine (2,3,4-trimethoxybenzyl-piperazine dihydrochloride) is a well-established drug that has been extensively used in the treatment of pathological conditions related with the generation of ROS, such as ischemia/reperfusion, heart surgery, brain disorders, and others. Trimetazidine is believed to exert its antioxidant effects as an inhibitor of ROS formation (Guamieri and Muscari, Biochem Pharmacol 1988, 37, 4685-88; Gartaoux et al., Emerit, I., ed. Antioxidants in therapy and preventive medicine. New York: Plenum Press; 1990: 383-88; Tsimoyiannis et al., Eur J Surg 1993, 159, 89-93; and Tetik et al., Trnanpl. Int. 1999, 12, 108-112), and as a metal chelator (Tselepis et al., Free Radical Biology & Medicine, 2001, 30(12), 1357-1364). Trimetazidine preserves intracellular phosphocreatine and adenosine triphosphate levels (Fragasso et al., J Am College Cardiology 2006, 48(5), 992-998) and affects myocardial substrate use by inhibiting oxidative phosphorylation and by shifting energy production from FFAs to glucose oxidation by selectively blocking long chain 3-ketoacyl coenzyme A thiolase activity, the last enzyme involved in FFA β-oxidation (Kantor et al., Circ Res 2000, 86, 580-8). By inhibiting fatty acid oxidation, trimetazidine, improves myocardial glucose utilization both at rest and during ischemia (Rosano et al., Cardiovascular Diabetology 2003, 2, 16; Kantor et al., Circ Res 2000, March 17, 580-588; and Rosano et al., Am J Cardiol 2006, 98[suppl], 14J-18J).

Propofol is known to inhibit or limit lipid peroxidation in cell membranes at clinically relevant concentrations (Bao et al., Br J Anaesthesia, 1998, 81, 584-589). For example, in a study examining the concentration of propofol required to inhibit mitochondrial peroxidation products, Eriksson, et al. demonstrated that propofol can inhibit fatty acid oxidation in mitochondria at concentrations as low as 0.1 μM or 0.02 μg/mL (Eriksson et al., Biochem Pharmacology 1992, 44(2), 391-393).

The efficacy of compounds provided by the present disclosure for treating cardiovascular diseases can be assessed using animal models and in clinical trials. Examples of rodent models of heart failure are described, for example, in Balakumar et al., J Pharmacological Toxicological Methods 2007, 56, 1-10.

### Neurological Diseases

Neurological diseases and disorders include neurodegenerative disorders such as Alzheimer's disease, Parkinson's disease, amyotrophic lateral sclerosis, mild cognitive impairment, Huntington's disease, multiple sclerosis, and cerebral ischemia; neuromuscular diseases such as amyotrophic lateral sclerosis, muscular dystrophies and myopathies, myasthenia gravis, post-polio syndrome, polymyositis, dermatomyositis, and inclusion body myositis, and neuropathies such as diabetic neuropathy, polyneuropathy, autonomic neuropathy, mononeuropathy, and mononeuritis multiplex.

A selective or a general loss of neurons is responsible for many acute or chronic neurological disorders. These pathophysiological situations, such as cerebral ischemia, involve an enhanced formation of free radicals in brain tissue. Both reactive oxygen species (e.g., superoxide *O₂⁻) and reactive nitrogen species (e.g., NO*) participate in the inflammatory process and contribute to neuronal death. NO* reacts rapidly with *O₂⁻ in aqueous media to form the highly reactive peroxyntirite (ONOO⁻) with harmful effects on neuronal cells. For example, oxidative stress is a contributing factor to neuropathic disorders such as Alzheimer's disease, Parkinson's disease, and CNS ischemia/reoxygenation injury (Halliwell, FASEB J 1987, 1, 358-364; and Lewen, J. Neurotrauma 2000, 17(10), 871-890).

Propofol exhibits neuroprotective effects on damage to cerebral neurons induced by forebrain ischemia (Ito et al., Acta Anaesthesiol Scand. 1999, 43(2), 153-62), in rat model of ischemia reperfusion injury (Young et al., Eur J Anaesthesiol 1997, 14(3), 320-6), antioxidant in inhibiting kainic acid induced lipid peroxidation in mouse brain homogenates (Lee et al., J Neurosurg Anesthesiol 2005, 17(3), 144-148), in cerebral ischemia (Ito et al., Acta Anaesthesiol Scand 1999, 43(2), 153-62; Kawaguchi et al., J. Anesth 2005, 19(2), 150-6; Adembri et al., Anesthesiology 2006, 1004, 80-89; Auvin et al., Bioorganic & Medicinal Chemistry Letters 2003, 13, 209-212; and Wilson and Gelb, J Neurosurg Anesth 2002, 14(1), 66-79), and against injuries caused by ischemia/reoxygenation (Young et al., Eur J Anaesthesol 1997, 14, 320-326; and De La Cruz et al., Anesth Analg 1998, 87, 1141-1146). Neuroprotection by propofol is in part attributed to its scavenging effect on peroxynitrite (Acquaviva et al., Anesthesiology 2004, 101(6), 1363-71). Propofol also exhibits neuroprotective effects in cerebral ischemia independent of its effect on low molecular weight antioxidants (Bayona et al., Anesthesiology 2004, 100, 1151-9), and in an in vitro model of oxygen-glucose deprivation possibly mediated by GLT1-independent restoration of glutamate uptake (Velly et al., Anesthesiology 2003, 99, 368-75).

Neurodegenerative diseases featuring cell death can be categorized as acute, i.e., stroke, traumatic brain injury, spinal cord injury, and chronic, i.e., amyotrophic lateral sclerosis, mild cognitive impairment, Huntington's disease, Parkinson's disease, and Alzheimer's disease. Although these diseases have different causes and affect different neuronal populations, they share similar impairment in intracellular energy metabolism. For example, the intracellular concentration of ATP is decreased, resulting in cystolic accumulation of Ca²⁺ and stimulation of formation of reactive oxygen species. Ca²⁺ and reactive oxygen species, in turn, trigger apoptotic cell death. The importance of NOS in neurodegenerative diseases is also recognized (Pannu and Singh, Neurochemistry International 2006, 49, 170-182). Oxidative stress is considered to play a role in the pathogenesis of neurodegenerative diseases such as Alzheimer's disease, mild cognitive impairment, Parkinson's disease, ALS, and Huntington's disease (see, e.g., Mariani et al., J Chromatogrpahy B 2005, 827, 65-75; and Espositio et al., Neurobiology of Aging 2003, 23, 719-735) and antioxidants show promise as neuroprotection in neurodegenerative disease (Moosmann and Behl, Expert Opin Investig Drugs 2002, 11(10), 1407-35; Casetta et al., Curr Pharm Des 2005, 11(16), 2033-52; and Sagara et al., J Neurochemistry 1999, 73(6), 2524-2530).

Parkinson's disease is a slowly progressive degenerative disorder of the nervous system characterized by tremor when muscles are at rest (resting tremor), slowness of voluntary movements, and increased muscle tone (rigidity). In Parkinson's disease, nerve cells in the basal ganglia, e.g., substantia nigra, degenerate, reducing the production of dopamine and the number of connections between nerve cells in the basal ganglia. As a result, the basal ganglia are unable to smooth muscle movements and coordinate changes in posture as normal, leading to tremor, incoordination, and slowed, reduced movement (bradykinesia). It is believed that oxidative stress may be a factor in the metabolic deterioration seen in Parkinson's disease tissue (Ebadi et al., Prog Neurobiol 1996, 48, 1-19; Jenner and Olanow, Ann Neurol 1998, 44 Suppl 1, S72-S84; and Sun and Chen, J Biomed Sci 1998, 5, 401-414).

The efficacy of administering a compound provided by the present disclosure for treating Parkinson's disease may be assessed using animal and human models of Parkinson's disease and clinical studies. Animal and human models of Parkinson's disease are known (see, e.g., O'Neil et al., CNS Drug Rev. 2005, 11(1), 77-96; Faulkner et al., Ann. Pharmacother. 2003, 37(2), 282-6; Olson et al., Am. J. Med. 1997, 102(1), 60-6; Van Blercom et al., Clin Neuropharmacol. 2004, 27(3), 124-8; Cho et al., Biochem. Biophys. Res. Commun. 2006, 341, 6-12; Emborg, J. Neuro. Meth. 2004, 139, 121-143; Tolwani et al., Lab Anim Sci 1999, 49(4), 363-71; Hirsch et al., J Neural Transm Suppl 2003, 65, 89-100; Orth and Tabrizi, Mov Disord 2003, 18(7), 729-37; Betarbet et al., Bioessays 2002, 24(4), 308-18; and McGeer and McGeer, Neurobiol Aging 2007, 28(5), 639-647). The ability of a compound provided by the present disclosure to mitigate against L-dopa induced dyskinesias can be assessed using, for example, animal models described in Lundblad et al., Experimental Neurology 2005, 194, 66-75; and Johnston et al., Experimental Neurology 2005, 191, 243-250.

Alzheimer's disease is a progressive loss of mental function characterized by degeneration of brain tissue, including loss of nerve cells and the development of senile plaques and neurofibrillary tangles. In Alzheimer's disease, parts of the brain degenerate, destroying nerve cells and reducing the responsiveness of the maintaining neurons to neurotransmitters. Abnormalities in brain tissue consist of senile or neuritic plaques, e.g., clumps of dead nerve cells containing an abnormal, insoluble protein called amyloid, and neurofibrillary tangles, twisted strands of insoluble proteins in the nerve cell. It is believed that oxidative stress may be a factor in the metabolic deterioration seen in Alzheimer's disease tissue with creatine kinase being one of the targets of oxidative damage (Pratico et al., FASEB J 1998, 12, 1777-1783; Smith et al., J Neurochem 1998, 70, 2212-2215; Yatin et al., Neurochem Res 1999, 24, 427-435; and Gilgun-Sherki et al., J Mol Neurosci 2003, 21(1), 1-11).

The efficacy of a compound provided by the present disclosure for treating Alzheimer's disease may be assessed using animal and human models of Alzheimer's disease and clinical studies. Useful animal models for assessing the efficacy of compounds for treating Alzheimer's disease are disclosed, for example, in Van Dam and De Dyn, Nature Revs Drug Disc 2006, 5, 956-970; Simpkins et al., Ann N Y Acad Sci, 2005, 1052, 233-242; Higgins and Jacobsen, Behav Pharmacol 2003, 14(5-6), 419-38; Janus and Westaway, Physiol Behav 2001, 73(5), 873-86; Bardgett et al., Brain Res Bull 2003, 60, 131-142; and Conn, ed., “Handbook of Models in Human Aging,” 2006, Elsevier Science & Technology.

Huntington's disease is an autosomal dominant neurodegenerative disorder in which specific cell death occurs in the neostriatum and cortex (Martin, N Engl J Med 1999, 340, 1970-80, which is incorporated by reference herein in its entirety). Onset usually occurs during the fourth or fifth decade of life, with a mean survival at age onset of 14 to 20 years. Huntington's disease is universally fatal, and there is no effective treatment. Symptoms include a characteristic movement disorder (Huntington's chorea), cognitive dysfunction, and psychiatric symptoms. The disease is caused by a mutation encoding an abnormal expansion of CAG-encoded polyglutamine repeats in the protein, huntingtin. A number of studies suggest that there is a progressive impairment of energy metabolism, possibly resulting from mitochondrial damage caused by oxidative stress as a consequence of free radical generation.

The efficacy of administering a compound provided by the present disclosure for treating Huntington's disease may be assessed using animal and human models of Huntington's disease and clinical studies. Animal models of Huntington's disease are disclosed, for example, in Riess and Hoersten, U.S. Application Publication No. 2007/0044162; Rubinsztein, Trends in Genetics, 2002, 18(4), 202-209; Matthews et al., J. Neuroscience 1998, 18(1), 156-63; Tadros et al., Pharmacol Biochem Behav 2005, 82(3), 574-82, and in Kaddurah-Daouk et al., U.S. Pat. No. 6,706,764 and U.S. Application Publication Nos. 2002/0161049, 2004/0106680, and 2007/0044162. An example of a placebo-controlled clinical trial evaluating the efficacy of a compound to treat Huntington's disease is disclosed in Verbessem et al., Neurology 2003, 61, 925-230.

Amyotrophic lateral sclerosis (ALS) is a progressive neurodegenerative disorder characterized by the progressive and specific loss of motor neurons in the brain, brain stem, and spinal cord (Rowland and Schneider, N Engl J Med 2001, 344, 1688-1700, which is incorporated by reference herein in its entirety). ALS begins with weakness, often in the hands and less frequently in the feet that generally progress up an arm or leg. Over time, weakness increases and spasticity develops characterized by muscle twitching and tightening, followed by muscle spasms and possibly tremors.

The efficacy a compound of a compound provided by the present disclosure for treating ALS may be assessed using animal and human models of ALS and clinical studies. Natural disease models of ALS include mouse models (motor neuron degeneration, progressive motor neuropathy, and wobbler) and the hereditary canine spinal muscular atrophy canine model (Pioro and Mitsumoto, Clin Neurosci 1995-1996, 3(6), 375-85). Experimentally produced and genetically engineered animal models of ALS can also useful in assessing therapeutic efficacy (see e.g., Doble and Kennelu, Amyotroph Lateral Scler Other Motor Neuron Disord. 2000, 1(5), 301-12; Grieb, Folia Neuropathol. 2004, 42(4), 239-48; Price et al., Rev Neurol (Paris) 1997, 153(8-9), 484-95; and Klivenyi et al., Nat Med 1999, 5, 347-50). Specifically, the SOD1-G93A mouse model is a recognized model for ALS. Examples of clinical trial protocols useful in assessing treatment of ALS are described, for example, in Mitsumoto, Amyotroph Lateral Scler Other Motor Neuron Disord. 2001, 2 Suppl 1, S10-S14; Meininger, Neurodegener Dis 2005, 2, 208-14; and Ludolph and Sperfeld, Neurodegener Dis. 2005, 2(3-4), 215-9.

Multiple sclerosis (MS) is an immune-mediated disease with inflammation and neurodegeneration contributing to neuronal demyelination and axonal injury. There is increasing evidence that oxidative stress is an important component in the pathogenesis of multiple sclerosis with excess ROS generated by macrophages and weakened cellular antioxidant defenses in the CNS leading to neuroal cell death (Gilgun-Sherki et al., J Neurol 2004, 251(3), 261-68; and Carlson and Rose, CNS Drugs 2006, 20(6), 433-41).

Assessment of MS treatment efficacy in clinical trials can be accomplished using tools such as the Expanded Disability Status Scale (Kurtzke, Neurology 1983, 33, 1444-1452) and the MS Functional Composite (Fischer et al., Mult Scle, 1999, 5, 244-250) as well as magnetic resonance imaging lesion load, biomarkers, and self-reported quality of life (see e.g., Kapoor, Cur Opinion Neurol 2006, 19, 255-259). Animal models of MS shown to be useful to identify and validate potential therapeutics include experimental autoimmune/allergic encephalomyelitis (EAE) rodent models that simulate the clinical and pathological manifestations of MS (Werkerle and Kurschus, Drug Discovery Today: Disease Models, Nervous System Disorders 2006, 3(4), 359-367; Gijbels et al., Neurosci Res Commun 2000, 26, 193-206; and Hofstetter et al., J Immunol 2002, 169, 117-125; Peiris et al., J Neuroscience Methods 2007, 163, 245-254; Kanwar, Curr med Chem 2005, 12(25), 2947-62; Ransohoff, J Clin Invest 2006, 116(9), 2313-2316; and Freedman, in “Advances in Neurology,” vol. 98, Lippincott Williams & Wilkins, 2006), and nonhuman primate EAE models ('t Hart et al., Immunol Today 2000, 21, 290-297).

Diabetic neuropathy is a common complication of diabetes mellitus in which nerves are damaged as a result of hyperglycemia. One of the most promising approaches for intervention and halting of diabetic neuropathy is the prevention of oxidative stress (Busui et al., Diabetes Metab Res Rev 2006, 22, 257-273; and Malik, Treat Endocrinol 2003, 2(6), 389-400). A variety of antioxidants including vitamin E have been demonstrated to have beneficial effects in treating diabetic neuropathy in diabetes patients and diabetic animal models (Manzella et al., Am J Clin Nutr 2001, 73, 1052-1057; van Dam et al., Eur. J. Pharmacol 1999, 376, 217-222; and Nicklander et al., J Neurol Sci 1994, 126, 6-14).

The efficacy of compounds provided by the present disclosure for treating diabetic neuropathy can be assessed using animal models and in clinical trials. Examples of mouse models of diabetic neuropathy are described, for example, in Sullivan et al., Neurobiol Dis 2007, dol: 10.1016/j.nbd.2007.07.022; and also see Animal Models of Diabetic Complications Consortium (NIH).

### Liver Diseases

Oxidative is a common pathogenetic mechanism contribution to initiation and progression of hepatic damage and a variety of liver discords such as alcoholic liver disease, chronic viral hepatitis, autoimmune liver diseases, and non-alcoholic steatohepatitis. Non-alcoholic fatty liver disease represents a spectrum of liver diseases, characterized mainly by macrovesicular steatosis in the absence of significant alcohol ingestion. Non-alcoholic fatty liver disease includes both non-alcoholic fatty liver diseases (NAFLD) and non-alcoholic steatohepatitis (NASH) (Comar and Sterling, Aliment Pharmacol Ther 2006, 23(2), 207-15; Charlton, Clin Gastroenterol Hepatol 2004, 2(12), 1048-58; and Portincasa et al., Clin Biochem 2005, 38, 203-217). NASH can lead to progressive fibrosis and cirrhosis. It is recognized that non-hepatic mechanisms are largely responsible for the development of insulin resistance, which causes hepatic steatosis, however, once developed oxidative stress and diminished antioxidants within the liver initiate the progression from steatosis to NASH and cirrhosis (McCullough, J Clin Gastroenterol 2006, 40(3 Suppl 1), S17-29; Albano et al., Aliment Pharmacol Ther 2005, 22(Nov Suppl 2), S71-73; and Contos and Sanyal, Adv Anat Pathol 2002, 9(1), 37-51). Mitochondria generated ROS and the accumulation of excessive hepatic fat primarily due to insulin resistance are believed to be responsible for the progression of NASH (Mehta et al., Nutr Rev. 2002, 60(9), 289-93).

The use of antioxidants such as S-adenosylmethoionine, α-tocopherol, polyenylphosphatidylchole, silymarin, N-acetylcysteine, betaine, and others has been shown to be beneficial in the treatment of chronic liver diseases (Mehta et al., Nutr Rev 2002, 60(9), 289-93; Dryden et al., Curr Gastroenterol Rep 2005, 7(4), 308-16; Medina and Moreno-Otero, Drugs, 2005, 65(17), 2445-61; and Gawrieh et al., J Investig Med 2004, 52(8), 506-14). Thiazolidinediones, such as rosiglitazone and pioglitazone, have shown promise in the treatment of NASH and the efficacy of adjunctive therapy with antioxidants such as alpha tocopherol are being investigated (Harrison, Curr Gastroenterol Rep 2006, 8(1), 21-9; and Liangpunsakul and Chalasani, Curr Treat Options Gastroenterol, 2003, 6(6), 455-463). For example, combined administration of pioglitazone and α-tocopherol produced a significant increase in metabolic clearance of glucose and a decrease in fasting free fatty acid and insulin in patients with NASH compared to α-tocopherol alone (Sanyal et al., Clin Gastroenterol Hepatol 2004, 2(12), 1059-15).

The efficacy of compound provided by the present disclosure for treating liver diseases can be assessed using animal models and in clinical trials. Examples of animal models of NASH are disclosed in London and George, Clin Liver Dis 2007, 11(1), 55-74; Ibanez et al., J Gastroenterol Hepatol 2007, 22(6), 846-51; Koteish and Diehl, Semin Liver Dis 2001, 21, 89-104; and Otogawa and Kawada, Nippon Rinsho 2006, 64(4), 1043-47. Examples of animal models of fatty liver disease are disclosed in Kainuma et al., J Gastroenterol 2006, 41(10), 971-80; and Anstee and Goldin, Int J Pathol 2006, 87(1), 1-16.

### Pulmonary Diseases

Oxidative stress mediated by ROS and NOS has also been implicated in the pathogenesis of chronic inflammatory lung diseases such as asthma, chronic obstructive pulmonary fibrosis, idiopathic pulmonary fibrosis, pulmonary fibrosis, acute respiratory distress syndrome, interstitial lung diseases, bronchopulmonary dysplasia, and cystic fibrosis (see e.g., Ricciardolo et al., Eur J Pharmacol 2006, 533, 240-252 and Rahman et al., Eur J Pharmacol 2006, 533, 222-239). Although the precise role of oxidative stress in diseases such as pulmonary fibrosis is not well understood (see e.g., Kinnula et al., Am J Respir Crit. Care Med 2005, 172, 417-412; Mastuzzo et al., Monaldi Arch Chest Dis 2002, 57(3-4), 173-6; and Antoniou et al., Pulmonary Pharmacology & Therapeutics, 2006, 28(3), 496-504), administration of antioxidants such α-tocopherol shows protective effects in animal models (Deger et al., Cell Biochem Funct 2006 Sep. 18, PMID 16981217).

Cystic fibrosis is a hereditary disease that causes certain glands to produce abnormal secretions, resulting in tissue and organ damage, especially in the lungs and the digestive tract. Patients with cystic fibrosis exhibit elevated indicators of oxidative stress and it has been suggested that maintaining and/or restoring oxidative balance can be useful in treating the disease (see e.g., Back et al., Am J Clin Nutr 2004, 80, 374-84).

The efficacy of compound provided by the present disclosure for treating pulmonary diseases can be assessed using animal models and in clinical trials. For example, animal models of asthma are disclosed in Isenberg-Feig et al., Current Allergy and Asthma Reports 2003, 3(1), 70-78; Evaldsson et al., International Immunopharmacology 2007, 7, 1025-1032; Hyde et al., Eur Resp Rev 2006, 15, 122-135; Pauluhn and Mohr, Experimental Toxicologic Pathology 2005, 56, 203-234; and Kips et al., Eur Respir J 2003, 22, 374-382. Animal models of fibrotic disorders of the lung are disclosed in Cuzzocrea et al., Am J Physiology—Lung Cellular and Molecular Physiology 2007, 292(5), L1095-L1104; Yara et al., Clin Experimental Immunology 2001, 124(1), 77-85; and Hayashi et al., Toxicologic Pathology 1995, 23(1), 63-71

### Dose

The amount of a form of propofol that will be effective in the treatment of a particular disease, disorder, or condition disclosed herein will depend on the nature of the disease, disorder, or condition, and can be determined by standard clinical techniques known in the art. In addition, in vitro or in vivo assays may optionally be employed to help identify optimal dosage ranges. The amount of a compound administered can depend on, among other factors, the patient being treated, the weight of the patient, the health of the patient, the disease being treated, the severity of the affliction, the route of administration, the potency of the compound, and the judgment of the prescribing physician.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

In certain embodiments, a therapeutically effective dose of a form of propofol may comprise from about 1 mg-equivalents to about 2,000 mg-equivalents of propofol per day, from about 5 mg-equivalents to about 1000 mg-equivalents of propofol per day, and in certain embodiments, from about 10 mg-equivalents to about 500 mg-equivalents of propofol per day.

A dose may be administered in a single dosage form or in multiple dosage forms. When multiple dosage forms are used the amount of a form of propofol contained within each of the multiple dosage forms may be the same or different.

In certain embodiments, an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. A dose of a highly orally bioavailable form of propofol may be within a range of circulating concentrations in for example the blood, plasma, or central nervous system, that is therapeutically effective, that is less than a sedative dose, and that exhibits little or no toxicity. A dose may vary within this range depending upon the dosage form employed.

During treatment a dose and dosing schedule may provide sufficient or steady state systemic concentrations of a therapeutically effective amount of propofol to treat a disease. In certain embodiments, an escalating dose may be administered.

Forms of propofol that provide a high oral bioavailability of propofol may be administered orally, and may be administered at intervals for as long as necessary to obtain an intended or desired therapeutic effect.

### Combination Therapy

Forms of propofol that provide a high oral bioavailability of propofol may be used in combination therapy with at least one other therapeutic agent. Forms of propofol and other therapeutic agent(s) can act additively or, and in certain embodiments, synergistically. In some embodiments, forms of e propofol may be administered concurrently with the administration of another therapeutic agent, such as for example, a compound for treating a metabolic, cardiovascular, neurological, liver, or pulmonary disease. In some embodiments, forms of propofol may be administered prior or subsequent to administration of another therapeutic agent, such as for example, a compound for treating a metabolic, cardiovascular, neurological, liver, or pulmonary disease.

Methods provided by the present disclosure include administering one or more forms of propofol and one or more other therapeutic agents provided that the combined administration does not inhibit the therapeutic efficacy of the one or more forms of propofol and/or other therapeutic agent and/or does not produce adverse combination effects.

In certain embodiments, forms of propofol may be administered concurrently with the administration of another therapeutic agent, which may be part of the same pharmaceutical composition or dosage form as, or in a different composition or dosage form than that containing a form of propofol. When a form of propofol is administered concurrently with another therapeutic agent that potentially can produce adverse side effects including, but not limited to, toxicity, the therapeutic agent may be administered at a dose that falls below the threshold at which the adverse side effect is elicited.

In certain embodiments, forms of propofol may be administered prior or subsequent to administration of another therapeutic agent. In certain embodiments of combination therapy, the combination therapy comprises alternating between administering a form of propofol and a composition comprising another therapeutic agent, e.g., to minimize adverse side effects associated with a particular drug.

In certain embodiments, forms of propofol may be administered to a patient together with one or more drugs useful in treating a metabolic disease such as diabetes mellitus type I, diabetes mellitus type II, metabolic syndrome, hypertension, and/or obesity.

Drugs useful in treating diabetes mellitus type I include insulin and octreotide.

Drugs useful in treating diabetes mellitus type II include acarbose, chlorpropamide, glimepriride, glipizide, glyburide, insulin, metformin, miglitol, nateglinide, pioglitazone, repaglinide, and rosiglitazone.

Drugs useful in treating hyperlipidemia include aspirin, clofibrate, ezetimibe, fluvastatin, gemfibrozil, lovastatin, and simvastatin.

Drugs useful in treating hypertension include acebutolol, amiloride, amlodipine, atenolol, benazepril, betaxolol, bisoprolol, candesartan, captopril, carteolol, carvedilol, chlorothiazide, chlorthalidone, clonidine, diltiazem, doxazosin, enalapril, eplerenone, eprosartan, felodipine, fosinopril, furosemide, guanabenz, guanadrel, guanethidine, guanfacine, hydralazine, hydrochlorothiazide, indapamide, irbesartan, isradipine, labetalol, lisinopril, losartan, methyldopa, metolazone, metoprolol, minoxidil, moexipril, nadolol, nicardipine, nifedipine, nisoldipine, nitroglycerin, olmesartan, perindopril, pindolol, prazosin, propranolol, quinapril, ramipril, reserpine, spironolactone, telmisartan, terazosin, timolol, torsemide, trandolapril, valsartan, and verapamil.

Drugs useful in treating hypoglycemia include glucagon.

Drugs useful in treating obesity include diethylpropion, methamphetamine, orlistat, phendimetrazine, and sibutramine.

In certain embodiments, forms of propofol may be administered to a patient together with one or more drugs useful for treating a cardiovascular disease, such as congestive heart failure, myocardial infarction, pulmonary hypertension, hypertrophic cardiomyopathy, arrhythmias, aoritic stenosis, angina pectoris, cardiac arrhythmia, ischemic stroke, and ischemic cardiomyopathy.

Drugs useful in treating congestive heart failure include allopurinol, amlodipine, benazepril, bisoprolol, captopril, carvedilol, digoxin, diltiazem, enalapril, eplerenone, fosinopril, furosemide, hydralazine, hydrochlorothiazide, isosorbide dinitrate, isosorbide mononitrate, lisinopril, metoprolol, moexipril, nesiritide, nicardipine, nifedipine, nitroglycerin, perindopril, prazosin, quinapril, ramipril, spironolactone, torsemide, trandolapril, triamcinolone, and valsartan.

Drugs useful in treating myocardial infarction include aspirin, atenolol, clopidogrel, dalteparin, lisinopril, magnesium chloride, metoprolol, moexipril, nitroglycerin, perindopril, propranolol, ramipril, timolol, and trandolapril.

Drugs useful in treating pulmonary hypertension include bosentan, isosorbide dinitrate, and treprostinil.

Drugs useful in treating hypertrophic cardiomyopathy include nifedipine.

Drugs useful in treating arrhythmias include amiodarone, disopyramide, dofetilide, mexiletine, phenyloin, procainamide, propranolol, quinidine, tocamide, and verapamil.

Drugs useful in treating aortic stenosis include propranolol.

Drugs useful in treating angina pectoris include amlodipine, aspirin, atenolol, carvedilol, heparin, metoprolol, nadolol, nitroglycerin, propranolol, timolol, and verapamil.

Drugs useful in treating cardiac arrhythmia include isoproterenol.

Drugs useful in treating ischemic stroke include aspirin, nimodipine, clopidogrel, pravastatin, unfractionated heparin, eptifibatide, β-blockers, angiotensin-converting enzyme (ACE) inhibitors, and enoxaparin.

Drugs useful in treating ischemic cardiomyopathy or ischemic heart disease include ACE inhibitors such as ramipril, captopril, and lisinopril; β-blockers such as acebutolol, atenolol, betaxolol, bisoprolol, carteolol, nadolol, penbutolol, propranolol, timolol, metoprolol, carvedilol, and aldosterone; diuretics; and digitoxin.

In certain embodiments, other drugs useful for treating cardiovascular diseases include blood-thinners, cholesterol lowering agents, anti-platelet agents, vasodilators, β-blockers, angiotensin blockers, and digitalis and its derivatives.

In certain embodiments, forms of highly orally bioavailable propofol may be administered to a patient together with one or more compounds for treating a neurological disease such as Parkinson's disease, Alzheimer's disease, ALS, multiple sclerosis, Huntington's disease, and diabetic neuropathy.

Drugs useful in treating Parkinson's disease include amantadine, benztropine, bromocriptine, levodopa, pergolide, pramipexole, ropinirole, selegiline, and trihexyphenidyl.

Drugs useful in treating Alzheimer's disease include donepezil, galantamine, memantine, rivastigmine, tacrine, and vitamin E.

Drugs useful in treating ALS include riluzole.

Drugs useful in treating multiple sclerosis include azathioprine, glatiramer, mitoxantrone, and prednisolone.

Drugs useful in treating diabetic neuropathy include carbamazepine.

Drugs useful in treating Huntington's disease include creatine phosphate.

In certain embodiments, forms of propofol may be administered to a patient together with one or more compounds for treating a liver disease is chosen from alcoholic liver disease, chronic viral hepatitis, autoimmune liver diseases, and non-alcoholic steatohepatitis, and non-alcoholic fatty liver disease.

Drugs useful in treating alcoholic liver disease include oxandrolone and propylthiouracil.

Drugs useful in treating chronic viral hepatitis include alpha interferon, peginterferon, ribavirin, lamivudine, and adefovir dipivoxil.

Drugs useful in treating autoimmune liver diseases include prednisone and azathioprine.

Durgs useful in treating non-alcoholic steatohepatities include metformin and thiazolidinones such as pioglitazone, troglitizone, and rosiglitazone.

Drugs useful in treating non-alcoholic fatty liver disease (steatorrhoeic hepatosis) and non-alcoholic steatohepatitis include metformin and thiazolidinones such as pioglitazone, troglitizone, and rosiglitazone.

Other drugs useful for treating liver diseases include telbivudine, entecavir, and protease inhibitors such as telaprevir and other disclosed, for example, in Tung et al., U.S. Application Publication Nos. 2005/0148548, 2004/0167116, and 2003/0144217; and in Hale et al., U.S. Application Publication No. 2004/0127488.

In certain embodiments, forms of propofol may be administered to a patient together with one or more compounds for treating a pulmonary disease such as asthma, chronic obstructive pulmonary fibrosis, idiopathic pulmonary fibrosis, pulmonary fibrosis, acute respiratory distress syndrome, interstitial lung diseases, bronchopulmonary dysplasia, and cystic fibrosis.

Drugs useful in treating asthma include flunisolide, metaproterenol, methylprednisolone, prednisone, triamcinolone, albuterol, aminophylline, bitolterol, epinephrine, hydrocortisone, isoproterenol, levalbuterol, pirbuterol, terbutaline, theophylline, beclomethasone, budesonide, cromolyn sodium, fluticasone, formoterol, levalbuterol, motelukast, nedocromil, omalizumab, oxtriphylline, pirbuterol, salmeterol, zafirlukast, and zileuton.

Drugs useful in treating pulmonary fibrosis include infliximab.

Drugs useful in treating idiopathic pulmonary fibrosis include interferon γ-lb.

Drugs useful in treating chronic obstructive pulmonary disease include metaproterenol, albuterol, bitolterol, fluticasone, formoterol, ipratropium, levalbuterol, pirbuterol, and salmeterol.

Drugs useful in treating acute respiratory distress syndrome include antibiotics, nitric oxide, and corticosteroids such as methylprednisolone.

Drugs useful in treating bronchopulmonary dysplasia include corticosteroids, bronchodilators, and surfactants.

Drugs useful in treating cystic fibrosis include amikacin, doruase alfa, gentamicin, ibuprofen, vitamin E, hyperonic saline, acetyl cysteine, albuterol, ipratropium bromide, and antibiotics such as vanomycin, tobramycin, meropenem, ciprofloxacin, piperacillin, colistin, and azithromycin.

### EXAMPLES

The following examples describe in detail methods of using forms of propofol that provide a high oral bioavailability of propofol. It will be apparent to those skilled in the art that many modifications, both to materials and methods, may be practiced without departing from the scope of the disclosure.

### Example 1

### Pharmacokinetics of Compound (2) and Propofol in Rats

Propofol or compound (2) was administered as an intravenous bolus injection or by oral gavage to groups of four to six adult male Sprague-Dawley rats (about 250 g). Animals were conscious at the time of the experiment. When orally administered, propofol or compound (2) was administered as an aqueous solution at a dose equivalent to propofol per kg body weight. When administered intravenously, propofol was administered as a solution (Diprivan®, Astra-Zeneca) at a dose equivalent to 10 or 15 mg of propofol per kg body weight. Animals were fasted overnight before the study and for 4 hours post-dosing. Blood samples (0.3 mL) were obtained via a jugular vein cannula at intervals over 8 hours following oral dosing. Blood was quenched immediately using acetonitrile with 1% formic acid and then was frozen at −80° C. until analyzed.

Three hundred (300) μL of 0.1% formic acid in acetonitrile was added to blank 1.5 mL tubes. Rat blood (300 μL) was collected at different times into tubes containing EDTA and vortexed to mix. A fixed volume of blood (100 μL) was immediately added into the Eppendorf tube and vortexed to mix. Ten microliters of a propofol standard stock solution (0.04, 0.2, 1, 5, 25, and 100 μg/mL) was added to 90 μL of blank rat blood quenched with 300 μL of 0.1% formic acid in acetonitrile. Then, 20 μL of p-chlorophenylalanine was added to each tube to make a final calibration standard (0.004, 0.02, 0.1, 0.5, 2.5, and 10 μg/mL). Samples were vortexed and centrifuged at 14,000 rpm for 10 min. The supernatant was analyzed by LC/MS/MS.

An API 4000 LC/MS/MS spectrometer equipped with Agilent 1100 binary pumps and a CTC HTS-PAL autosampler and a Phenomenex Synergihydro-RP 4.6×30 mm column were used in the analysis. The mobile phase for propofol analysis was (A) 2 mM ammonium acetate, and (B) 5 mM ammonium acetate in 95% acetonitrile. The mobile phase for the analysis of compound (2) was (A) 0.1% formic acid, and (B) 0.1% formic acid in acetonitrile. The gradient condition was: 10% B for 0.5 min, then to 95% B in 2.5 min, then maintained at 95% B for 1.5 min. The mobile phase was returned to 10% B for 2 min. An APCI source was used on the API 4000. The analysis was done in negative ion mode for propofol and in positive ion mode for compound (2). The MRM transition for each analyte was optimized using standard solutions. Five (5) μL of each sample was injected. Non-compartmental analysis was performed using WinNonlin (v.3.1 Professional Version, Pharsight Corporation, Mountain View, Calif.) on individual animal profiles. Summary statistics on major parameter estimates was performed for Cₘₐₓ (peak observed concentration following dosing), Tₘₐₓ (time to maximum concentration is the time at which the peak concentration was observed), AUC₀₋ₜ (area under the serum concentration-time curve from time zero to last collection time, estimated using the log-linear trapezoidal method), AUCO_{0-∞}, (area under the serum concentration time curve from time zero to infinity, estimated using the log-linear trapezoidal method to the last collection time with extrapolation to infinity), and t_{1/2} (terminal half-life).

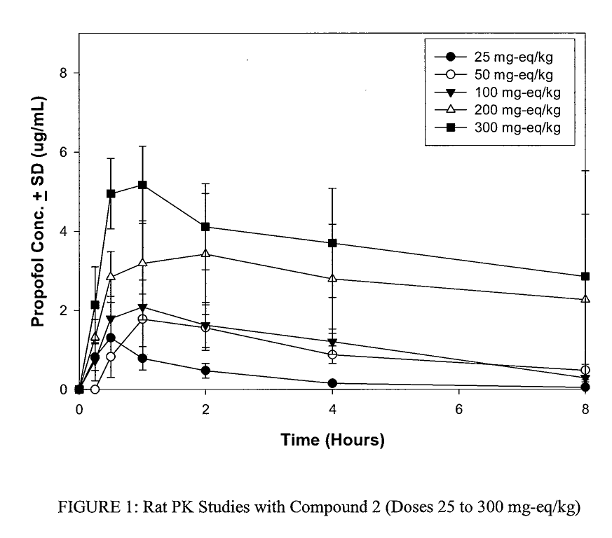

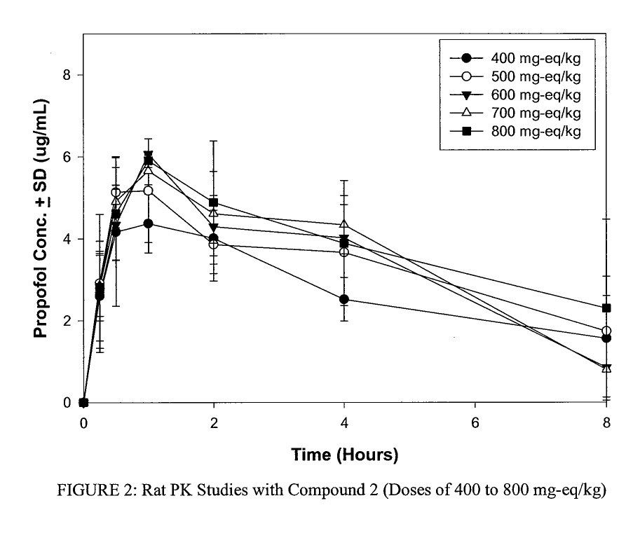

The oral bioavailability (F %) of propofol was determined by comparing the area under the propofol concentration vs time curve (AUC) following oral administration of compound (2) with the AUC of the propofol concentration vs time curve following intravenous administration of propofol on a dose normalized basis. The results from these studies are summarized in FIG. 1, FIG. 2, and Table 1.

TABLE 1
Pharmacokinetic Parameter Summary for Rat Study

| Compound | | | | | | | | | | |
|---|---|---|---|---|---|---|---|---|---|---|
| (2) | | | | | | | | | | |
| Dose Level | Cₘₐₓ | Tₘₐₓ | T_{1/2-1} | | AUCₜ | | AUC_{inf} | | Fₚₒ | |
| (mg-eq/kg) | (μg/mL) | (hr) | (hr) | | (hr · μg/mL) | | (hr · μg/mL) | | (%) | |
| 25 | 0.8 (0.2) | 1.7 (0.5) | 2.2 | (0.5) | 2.5 | (0.7) | 2.8 | (0.7) | 65 | (17) |
| 50 | 2.0 (0.8) | 2.0 (1.2) | 3.2 | (2.9) | 5.0 | (1.7) | 6.0 | (2.0) | 78 | (23) |
| 100 | 2.2 (0.4) | 1.1 (0.6) | 2.8 | (0.7) | 9.1 | (1.3) | 10.3 | (0.9) | 61 | (6) |
| 200 | 3.4 (2.0) | 1.3 (1.1) | 5.0 | (4.3) | 18.5 | (12.2) | 24.6 | (6.7) | 72 | (20) |
| 300 | 4.6 (0.7) | 0.8 (0.4) | 2.4 | (0.4) | 18.7 | (0.5) | 20.9 | (0.1) | 41 | (0) |
| 400 | 4.7 (0.7) | 1.0 (0.7) | 2.6 | (0.8) | 22.0 | (2.7) | 25.0 | (1.2) | 37 | (2) |
| 500 | 5.0 (0.6) | 2.3 (1.7) | 11.1 | (0.1) | 41.7 | (17.5) | 53.4 | (23.9) | 83 | (28) |
| 600 | 6.1 (0.0) | 1.0 (0.0) | 2.4 | (0.0) | 25.4 | (22.4) | 33.4 | (11.2) | 33 | (11) |
| 700 | 5.6 (0.3) | 1.0 (0.0) | 3.8 | (2.7) | 24.3 | (5.2) | 40.1 | (18.7) | 39 | (21) |
| 800 | 6.0 (0.5) | 1.3 (0.6) | 6.1 | (5.7) | 29.5 | (11.9) | 60.6 | (53.6) | 60 | (53) |

### Example 2

### Pharmacokinetics of Compound (2) and Propofol in Dogs

Compound (2) or propofol was administered by oral gavage or as an intravenous bolus injection, respectively, to groups of two to four adult male Beagle dogs (about 8 kg) as solutions in water. Animals were fasted overnight before the study and for 4 hours post-dosing. Blood samples (1.0 mL) were obtained via the femoral vein at intervals over 24 hours after oral dosing. Blood was quenched immediately using acetonitrile with 1% formic acid and then frozen at −80° C. until analyzed. Compound (2) was administered to dogs with a minimum of 7-day wash out period between dosing sessions.

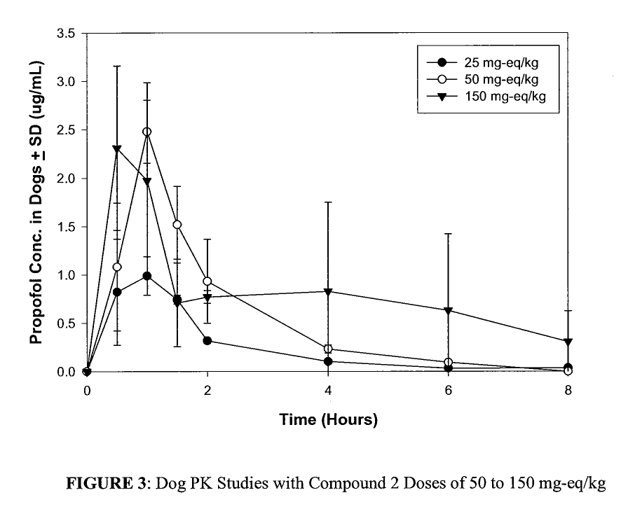

Bood sample preparation and LC/MS/MS analysis were the same as for the rat study described in Example 1. The pharmacokinetics of propofol following oral administration of compound (2) to dogs is summarized in FIG. 3 and Table 2.

TABLE 2
Pharmacokinetic Parameter Summary for Dog Study

| Compound | | | | | | | |
|---|---|---|---|---|---|---|---|
| (2) | | | | | | | |
| Dose Level | Cₘₐₓ | Tₘₐₓ | T_{1/2-1} | AUCₜ | AUC_{inf} | Fₚₒ | |
| (mg-eq/kg) | (μg/mL) | (hr) | (hr) | (hr · μg/mL) | (hr · μg/mL) | (%) | |
| 25 | 1.0 (0.3) | 0.8 (0.4) | 0.9 (0.1) | 1.8 (0.5) | 2.0 (0.5) | 37 | (10) |
| 50 | 2.5 (0.3) | 1.0 (0.0) | 1.1 (0.1) | 4.3 (0.7) | 4.4 (0.7) | 41 | (6) |
| 150 | 2.3 (0.8) | 0.5 (0.0) | 2.3 (0.6) | 6.7 (5.0) | 7.9 (6.5) | 25 | (20) |

### Example 3

### Toxicity Studies

Acute toxicity studies in rats were undertaken to assess the tolerance of a single oral dose of compound (2) formulated in water. The results indicated that compound (2) was well tolerated at levels from about 49 mg-eq/kg to about 1552 mg-eq/kg of administered compound. Transient hypoactivity was observed at doses from about 49 mg-eq/kg up to about 388 mg-eq/kg within about 30 minutes of dose and maintained up to 4 hours post dose. Sedation was observed at doses from about 582 mg-eq/kg up to about 970 mg-eq/kg within about 1.5 hours of dose and lasted up to 4 hours post dose. Anesthesia was observed at doses from about 1164 mg-eq/kg up to about 1552 mg-eq/kg within about 1 hour of dose and lasted up to about 2 hours post dose. Complete recovery from hypoactivity, sedation, and anesthesia occurred in all rats within about 8 hours after dose. Doses above about 1552 mg-eq/kg (about 800 mg-eq/kg of propofol) were not tested.

Acute toxicity studies were also performed by orally administering a single dose of compound (2) formulated in water to groups of male beagle dogs at doses from about 25 mg-eq/kg to about 150 mg-eq/kg. Results indicated that at these doses compound (2) was well tolerated in dogs. No sedation or anesthesia was observed at these doses.

Multiple dose studies in rats were performed by orally administering compound (2) formulated in water to groups of male rats at doses of 49 mg-eq/kg to 97 mg-eq/kg for a period of five days, by oral gavages administered once a day. No adverse effects were observed in the multiple dose studies. Results indicated that compound (2) was well tolerated by rats. No sedation or anesthesia was observed at these doses.

### Example 4

### Animal Models for Assessing Therapeutic Efficacy of Forms of Propofol for Treating Parkinson's Disease

### MPTP Induced Neurotoxicity

MPTP, or 1-methyl-4-phenyl-1,2,3,6-tetrahydropyridine is a neurotoxin that produces a Parkinsonian syndrome in both man and experimental animals. Studies of the mechanism of MPTP neurotoxicity show that it involves the generation of a major metabolite, MPP⁺, formed by the activity of monoamine oxidase on MPTP. Inhibitors of monoamine oxidase block the neurotoxicity of MPTP in both mice and primates. The specificity of the neurotoxic effects of MPP⁺ for dopaminergic neurons appears to be due to the uptake of MPP⁺ by the synaptic dopamine transporter. Blockers of this transporter prevent MPP⁺ neurotoxicity. MPP⁺ has been shown to be a relatively specific inhibitor of mitochondrial complex I activity, binding to complex I at the retenone binding site and impairing oxidative phosphorylation. In vivo studies have shown that MPTP can deplete striatal ATP concentrations in mice. It has been demonstrated that MPP⁺ administered intrastriatally to rats produces significant depletion of ATP as well as increased lactate concentration confined to the striatum at the site of the injections. Compounds that enhance ATP production can protect against MPTP toxicity in mice.

A form of propofol is administered to animals such as mice or rats for three weeks before treatment with MPTP. MPTP is administered at an appropriate dose, dosing interval, and mode of administration for 1 week before sacrifice. Control groups receive either normal saline or MPTP hydrochloride alone. Following sacrifice the two striate are rapidly dissected and placed in chilled 0.1 M perchloric acid. Tissue is subsequently sonicated and aliquots analyzed for protein content using a fluorometer assay. Dopamine, 3,4-dihydroxyphenylacetic acid (DOPAC), and homovanillic acid (HVA) are also quantified. Concentrations of dopamine and metabolites are expressed as nmol/mg protein.

Forms of propofol that protect against DOPAC depletion induced by MPTP, HVA, and/or dopamine depletion are neuroprotective and therefore can be useful for the treatment of Parkinson's disease.

### Haloperidol-Induced Hypolocomotion

The ability of a compound to reverse the behavioral depressant effects of dopamine antagonists such as haloperidol, in rodents and is considered a valid method for screening drugs with potential antiparkinsonian effects (Mandhane, et al., Eur. J. Pharmacol. 1997, 328, 135-141). Hence, the ability of a form of propofol to block haloperidol-induced deficits in locomotor activity in mice can be used to assess both in vivo and potential anti-Parkinsonian efficacy.

Mice used in the experiments are housed in a controlled environment and allowed to acclimatize before experimental use. One and one-half hour before testing, mice are administered 0.2 mg/kg haloperidol, a dose that reduces baseline locomotor activity by at least 50%. A test compound is administered 5-60 min prior to testing. The animals are then placed individually into clean, clear polycarbonate cages with a flat perforated lid. Horizontal locomotor activity is determined by placing the cages within a frame containing a 3×6 array of photocells interfaced to a computer to tabulate beam interrupts. Mice are left undisturbed to explore for 1 h, and the number of beam interruptions made during this period serves as an indicator of locomotor activity, which is compared with data for control animals for statistically significant differences.

### 6-Hydroxydopamine Animal Model

The neurochemical deficits seen in Parkinson's disease can be reproduced by local injection of the dopaminergic neurotoxin, 6-hydroxydopamine (6-OHDA) into brain regions containing either the cell bodies or axonal fibers of the nigrostriatal neurons. By unilaterally lesioning the nigrostriatal pathway on only one-side of the brain, a behavioral asymmetry in movement inhibition is observed. Although unilaterally-lesioned animals are still mobile and capable of self maintenance, the remaining dopamine-sensitive neurons on the lesioned side become supersensitive to stimulation. This is demonstrated by the observation that following systemic administration of dopamine agonists, such as apomorphine, animals show a pronounced rotation in a direction contralateral to the side of lesioning. The ability of compounds to induce contralateral rotations in 6-OHDA lesioned rats has been shown to be a sensitive model to predict drug efficacy in the treatment of Parkinson's disease.

Male Sprague-Dawley rats are housed in a controlled environment and allowed to acclimatize before experimental use. Fifteen minutes prior to surgery, animals are given an intraperitoneal injection of the noradrenergic uptake inhibitor desipramine (25 mg/kg) to prevent damage to nondopamine neurons. Animals are then placed in an anaesthetic chamber and anaesthetized using a mixture of oxygen and isoflurane. Once unconscious, the animals are transferred to a stereotaxic frame, where anesthesia is maintained through a mask. The top of the animal's head is shaved and sterilized using an iodine solution. Once dry, a 2 cm long incision is made along the midline of the scalp and the skin retracted and clipped back to expose the skull. A small hole is then drilled through the skull above the injection site. In order to lesion the nigrostriatal pathway, the injection cannula is slowly lowered to position above the right medial forebrain bundle at −3.2 mm anterior posterior, −1.5 mm medial lateral from the bregma, and to a depth of 7.2 mm below the duramater. Two minutes after lowering the cannula, 6-OHDA is infused at a rate of 0.5 μL/min over 4 min, to provide a final dose of 8 μg. The cannula is left in place for an additional 5 min to facilitate diffusion before being slowly withdrawn. The skin is then sutured shut, the animal removed from the sterereotaxic frame, and returned to its housing. The rats are allowed to recover from surgery for two weeks before behavioral testing.

Rotational behavior is measured using a rotameter system having stainless steel bowls (45 cm dia×15 cm high) enclosed in a transparent Plexiglas cover around the edge of the bowl and extending to a height of 29 cm. To assess rotation, rats are placed in a cloth jacket attached to a spring tether connected to an optical rotameter positioned above the bowl, which assesses movement to the left or right either as partial (45°) or full (360°) rotations.

To reduce stress during administration of a test compound, rats are initially habituated to the apparatus for 15 min on four consecutive days. On the test day, rats are given a test compound, e.g., a form of propofol. Immediately prior to testing, animals are given a subcutaneous injection of a subthreshold dose of apomorphine, and then placed in the harness and the number of rotations recorded for one hour. The total number of full contralatral rotations during the hour test period serves as an index of antiparkinsonian drug efficacy.

### L-Dopa Induced Dyskinesia

The ability of forms of propofol to mitigate the effects of L-dopa induced dyskinesia can be assessed using an animal model described, for example, by Johnston et al., Experimental Neurology 2005, 191, 243-250.

Male, Sprague-Dawley rats (250-300 g) are housed and maintained under standard conditions.

Reserpine (4 mg/kg) is administered under light isofluorane anesthesia. Eighteen hours following reserpine administration, the animals are placed into observation cages. Behavior is assessed using an automated movement detection system that includes dual layers of rectangular grids of sensors containing an array of 24 infrared beams surrounding the cage. Each beam break is registered an activity count and contributes to the assessment of a variety of different behavioral parameters depending on the location of the event and the timing of successive beam breaks. These parameters include” (1) horizontal activity, a measure of the number of beams broken on the lower level; (2) vertical activity, a measure of beams broken on the upper level.

In one experiment, immediately prior to commencing behavioral assessments, rats are injected with a combination of L-dopa methyl ester and carbidopa (or benserazide). In another study, to assess the effects of forms of propofol on L-dopa induced activity, animals are randomly assigned to groups. In each group, immediately following L-dopa/carbidopa administration, vehicle or form of propofol is administered. The behavior of normal, non-resperine-treated, animals is also assessed. Behavior of the animals in the different groups is monitored for at least 4 hours. Forms of propofol that reduce the L-dopa-induced locomotion in the reserpine-treated rats are potentially useful in treating Parkinson's disease and/or the symptoms associated with Parkinson's disease.

### Example 5

### Use of Clinical Trials to Assess the Efficacy of Forms of Propofol for Treating Parkinson's Disease

The following clinical study may be used to assess the efficacy of a compound in treating Parkinson's disease.

Patients with idiopathic PD fulfilling the Queen Square Brain Bank criteria (Gibb et al., JNeurol Neurosurg Psychiatry 1988, 51, 745-752) with motor fluctuations and a defined short duration GABA analog response (1.5-4 hours) are eligible for inclusion. Clinically relevant peak dose dyskinesias following each morning dose of their current medication are a further pre-requisite. Patients are also required to have been stable on a fixed dose of treatment for a period of at least one month prior to starting the study. Patients are excluded if their current drug regime includes slow-release formulations of L-Dopa, COMT inhibitors, selegiline, anticholinergic drugs, or other drugs that could potentially interfere with gastric absorption (e.g. antacids). Other exclusion criteria include patients with psychotic symptoms or those on antipsychotic treatment, patients with clinically relevant cognitive impairment, defined as MMS (Mini Mental State) score of less than 24 (Folstein et al., JPsychiatrRes 1975, 12, 189-198), risk of pregnancy, Hoehn & Yahr stage 5 in off-status, severe, unstable diabetes mellitus, and medical conditions such as unstable cardiovascular disease or moderate to severe renal or hepatic impairment. Full blood count, liver, and renal function blood tests are taken at baseline and after completion of the study.

A randomized, double blind, and cross-over study design is used. Each patient is randomized to the order in which either L-dopa or one of the two dosages of test compound, e.g., a form of propofol, is administered in a single-dose challenge in double-dummy fashion in three consecutive sessions. Randomization is by computer generation of a treatment number, allocated to each patient according to the order of entry into the study. All patients give informed consent.

Patients are admitted to a hospital for an overnight stay prior to administration of test compound the next morning on three separate occasions at weekly intervals. After withdrawal of all antiparkinsonian medication from midnight the previous day, test compound is administered at exactly the same time in the morning in each patient under fasting conditions.

Patients are randomized to the order of the days on which they receive placebo or test compound. The pharmacokinetics of a test compound can be assessed by monitoring plasma propofol concentration over time. Prior to administration, a 22 G intravenous catheter is inserted in a patient's forearm. Blood samples of 5 ml each are taken at baseline and 15, 30, 45, 60, 75, 90, 105, 120, 140, 160, 180, 210, and 240 minutes after administering a test compound or until a full off state has been reached if this occurs earlier than 240 minutes after drug ingestion. Samples are centrifuged immediately at the end of each assessment and stored deep frozen until assayed. Plasma propofol levels are determined by high-pressure liquid chromatography (HPLC). On the last assessment additional blood may be drawn for routine hematology, blood sugar, liver, and renal function.

For clinical assessment, motor function is assessed using UPDRS (United Parkinson's Disease Rating Scale) motor score and BrainTest (Giovanni et al., J Neurol Neurosurg Psychiatry 1999, 67, 624-629), which is a tapping test performed with the patient's more affected hand on the keyboard of a laptop computer. These tests are carried out at baseline and then immediately following each blood sample until patients reach their full on-stage, and thereafter at 3 intervals of 20 min, and 30 min intervals until patients reach their baseline off-status. Once patients reach their full on-state, video recordings are performed three times at 20 min intervals. The following mental and motor tasks, which have been shown to increase dyskinesia (Duriff et al., Mov Disord 1999, 14, 242-245), are monitored during each video session: (1) sitting still for 1 minute; (2) performing mental calculations; (3) putting on and buttoning a coat; (4) picking up and drinking from a cup of water; and (5) walking. Videotapes are scored using, for example, versions of the Goetz Rating Scale and the Abnormal Involuntary Movements Scale to document a possible increase in test compound induced dyskinesia.

Actual occurrence and severity of dyskinesia is measured with a Dyskinesia Monitor (Manson et al., J Neurol Neurosurg Psychiatry 2000, 68, 196-201). The device is taped to a patient's shoulder on their more affected side. The monitor records during the entire time of a challenging session and provides a measure of the frequency and severity of occurring dyskinesias.

Results can be analyzed using appropriate statistical methods.

### Example 6

### Animal Model for Assessing Therapeutic Efficacy of Forms of Propofol for Treating Alzheimer's Disease

Heterozygous transgenic mice expressing the Swedish AD mutant gene, hAPPK670N, M671L (Tg2576; Hsiao, Learning & Memory 2001, 8, 301-308) are used as an animal model of Alzheimer's disease. Animals are housed under standard conditions with a 12:12 light/dark cycle and food and water available ad libitum. Beginning at 9 months of age, mice are divided into two groups. The first two groups of animals receive increasing doses of a form of propofol over six weeks. The remaining control group receives daily saline injections for six weeks.

Behavioral testing is performed at each drug dose using the same sequence over two weeks in all experimental groups: 1) spatial reversal learning, 2) locomotion, 3) fear conditioning, and 4) shock sensitivity. This order is selected to minimize interference among testing paradigms.

Acquisition of the spatial learning paradigm and reversal learning are tested during the first five days of test compound administration using a water T-maze as described in Bardgett et al., Brain Res Bull 2003, 60, 131-142. Mice are habituated to the water T-maze during days 1-3, and task acquisition begins on day 4. On day 4, mice are trained to find the escape platform in one choice arm of the maze until 6 to 8 correct choices are made on consecutive trails. The reversal learning phase is then conducted on day 5. During the reversal learning phase, mice are trained to find the escape platform in the choice arm opposite from the location of the escape platform on day 4. The same performance criterion and inter-trial interval are used as during task acquisition.

Large ambulatory movements are assessed to determine that the results of the spatial reversal learning paradigm are not influenced by the capacity for ambulation. After a rest period of two days, horizontal ambulatory movements, excluding vertical and fine motor movements, are assessed in a chamber equipped with a grid of motion-sensitive detectors on day 8. The number of movements accompanied by simultaneous blocking and unblocking of a detector in the horizontal dimension are measured during a one-hour period.

The animals' capacity for contextual and cued memory is tested using a fear conditioning paradigm beginning on day 9. Testing takes place in a chamber that contains a piece of absorbent cotton soaked in an odor-emitting solution such as mint extract placed below the grid floor. A 5-min, 3 trial 80 db, 2800 Hz tone-foot shock sequence is administered to train the animals on day 9. On day 10, memory for context is tested by returning each mouse to the chamber without exposure to the tone and foot shock, and recording the presence or absence of freezing behavior every 10 seconds for 8 minutes. Freezing is defined as no movement, such as ambulation, sniffing or stereotypy, other than respiration.

On day 11, the animals' response to an alternate context and to the auditory cue is tested. Coconut extract is placed in a cup and the 80 dB tone is presented, but no foot shock is delivered. The presence or absence of freezing in response to the alternate context is then determined during the first 2 minutes of the trial. The tone is then presented continuously for the remaining 8 minutes of the trial, and the presence or absence of freezing in response to the tone is determined.

On day 12, the animals are tested to assess their sensitivity to the conditioning stimulus, i.e., foot shock.

Following the last day of behavioral testing, animals are anesthetized and the brains removed, post-fixed overnight, and sections cut through the hippocampus. The sections were stained to image β-amyloid plaques (see e.g., Dong et al., Neuroscience 2004, 127, 601-609).

Data are analyzed using appropriate statistical methods.

### Example 7

### Animal Model for Assessing Therapeutic Efficacy of Forms of Propofol for Treating Huntington's Disease

### Neuroprotective Effects in a Transgenic Mouse Model of Huntington's Disease

Transgenic HD mice of the N171-82Q strain and non-transgenic littermates are treated with a prodrug form of propofol or a vehicle from 10 weeks of age. The mice are placed on a rotating rod (“rotarod”). The length of time at which a mouse falls from the rotarod is recorded as a measure of motor coordination. The total distance traveled by a mouse is also recorded as a measure of overall locomotion. Mice administered a form of propofol that is neuroprotective in the N171-82Q transgenic HD mouse model remain on the rotarod for a longer period of time and travel further than mice administered vehicle.

### Malonate Model of Huntington's Disease

A series of reversible and irreversible inhibitors of enzymes involved in energy generating pathways has been used to generate animal models for neurodegenerative diseases such as Parkinson's and Huntington's diseases. In particular, inhibitors of succinate dehydrogenase, an enzyme that impacts cellular energy homeostasis, has been used to generate a model for Huntington's disease (Brouillet et al., J. Neurochem. 1993, 60, 356-359; Beal et al., J. Neurosci. 1993, 13, 4181-4192; Henshaw et al., Brain Research 1994, 647, 161-166; and Beal et al., J. Neurochem. 1993, 61, 1147-1150). The enzyme succinate dehydrogenase plays a central role in both the tricarboxylic acid cycle as well as the electron transport chain in mitochondria. Malonate is a reversible inhibitor of succinate dehydrogenase. Intrastriatal injections of malonate in rats have been shown to produce dose dependent striatal excitotoxic lesions that are attenuated by both competitive and noncompetitive NMDA antagonists (Henshaw et al., Brain Research 1994, 647, 161-166). For example, the glutamate release inhibitor, lamotrigine, also attenuates the lesions. Co-injection with succinate blocks the lesions, consistent with an effect on succinate dehydrogenase. The lesions are accompanied by a significant reduction in ATP levels as well as a significant increase in lactate levels in vivo as shown by chemical shift resonance imaging (Beal et al., J. Neurochem. 1993, 61, 1147-1150). The lesions produce the same pattern of cellular sparing, which is seen in Huntington's disease, supporting malonate challenge as a useful model for the neuropathologic and neurochemical features of Huntington's disease.

To evaluate the effect of a form of propofol in this malonate model for Huntington's disease, a form of propofol is administered at an appropriate dose, dosing interval, and route, to male Sprague-Dawley rats. A prodrug is administered for two weeks prior to the administration of malonate and then for an additional week prior to sacrifice. Malonate is dissolved in distilled deionized water and the pH adjusted to 7.4 with 0.1 M HCl. Intrastriatal injections of 1.5 μL of 3 μmol malonate are made into the left striatum at the level of the Bregma 2.4 mm lateral to the midline and 4.5 mm ventral to the dura. Animals are sacrificed at 7 days by decapitation and the brains quickly removed and placed in ice cold 0.9% saline solution. Brains are sectioned at 2 mm intervals in a brain mold. Slices are then placed posterior side down in 2% 2,3,5-tiphenyltetrazolium chloride. Slices are stained in the dark at room temperature for 30 min and then removed and placed in 4% paraformaldehyde pH 7.3. Lesions, noted by pale staining, are evaluated on the posterior surface of each section. The measurements are validated by comparison with measurements obtained on adjacent Nissl stain sections. Compounds exhibiting a neuroprotective effect and therefore potentially useful in treating Huntington's disease show a reduction in malonate-induced lesions.

### Example 8

### Animal Model for Assessing Therapeutic Efficacy of Forms of Propofol for Treating Amyotrophic Lateral Sclerosis

A murine model of SOD1 mutation-associated ALS has been developed in which mice express the human superoxide dismutase (SOD) mutation glycine→alanine at residue 93 (SOD1). These SOD1 mice exhibit a dominant gain of the adverse property of SOD, and develop motor neuron degeneration and dysfunction similar to that of human ALS (Gurney et al., Science 1994, 264(5166), 1772-1775; Gurney et al., Ann. Neurol. 1996, 39, 147-157; Gurney, J. Neurol. Sci. 1997, 152, S67-73; Ripps et al., Proc Natl Acad Sci U.S.A. 1995, 92(3), 689-693; and Bruijn et al., Proc Natl Acad Sci U.S.A. 1997, 94(14), 7606-7611). The SOD1 transgenic mice show signs of posterior limb weakness at about 3 months of age and die at 4 months. Features common to human ALS include astrocytosis, microgliosis, oxidative stress, increased levels of cyclooxygenase/prostaglandin, and, as the disease progresses, profound motor neuron loss.

Studies are performed on transgenic mice overexpressing human Cu/Zn-SOD G93A mutations ((B6SJL-TgN(SOD 1-G93A) 1 Gur)) and non-transgenic B6/SJL mice and their wild litter mates. Mice are housed on a 12-hr day/light cycle and (beginning at 45 d of age) allowed ad libitum access to either test compound-supplemented chow, or, as a control, regular formula cold press chow processed into identical pellets. Genotyping can be conducted at 21 days of age as described in Gurney et al., Science 1994, 264(5166), 1772-1775. The SOD1 mice are separated into groups and treated with a test compound, e.g., a form of propofol, or serve as controls.

The mice are observed daily and weighed weekly. To assess health status mice are weighed weekly and examined for changes in lacrimation/salivation, palpebral closure, ear twitch and pupillary responses, whisker orienting, postural and righting reflexes and overall body condition score. A general pathological examination is conducted at the time of sacrifice.

Motor coordination performance of the animals can be assessed by one or more methods known to those skilled in the art. For example, motor coordination can be assessed using a neurological scoring method. In neurological scoring, the neurological score of each limb is monitored and recorded according to a defined 4-point scale: O-normal reflex on the hind limbs (animal will splay its hind limbs when lifted by its tail); 1—abnormal reflex of hind limbs (lack of splaying of hind limbs weight animal is lifted by the tail); 2—abnormal reflex of limbs and evidence of paralysis; 3—lack of reflex and complete paralysis; and 4—inability to right when placed on the side in 30 seconds or found dead. The primary end point is survival with secondary end points of neurological score and body weight. Neurological score observations and body weight are made and recorded five days per week. Data analysis is performed using appropriate statistical methods.

The rotarod test evaluates the ability of an animal to stay on a rotating dowel allowing evaluation of motor coordination and proprioceptive sensitivity. The apparatus is a 3 cm diameter automated rod turning at, for example, 12 rounds per min. The rotarod test measures how long the mouse can maintain itself on the rod without falling. The test can be stopped after an arbitrary limit of 120 sec. Should the animal fall down before 120 sec, the performance is recorded and two additional trials are performed. The mean time of 3 trials is calculated. A motor deficit is indicated by a decrease of walking time.

In the grid test, mice are placed on a grid (length: 37 cm, width: 10.5 cm, mesh size: 1×1 cm²) situated above a plane support. The number of times the mice put their paws through the grid is counted and serves as a measure for motor coordination.

The hanging test evaluates the ability of an animal to hang on a wire. The apparatus is a wire stretched horizontally 40 cm above a table. The animal is attached to the wire by its forepaws. The time needed by the animal to catch the string with its hind paws is recorded (60 sec max) during three consecutive trials.

Electrophysiological measurements (EMG) can also be used to assess motor activity condition. Electromyographic recordings are performed using an electromyography apparatus. During EMG monitoring mice are anesthetized. The measured parameters are the amplitude and the latency of the compound muscle action potential (CMAP). CMAP is measured in gastrocnemius muscle after stimulation of the sciatic nerve. A reference electrode is inserted near the Achilles tendon and an active needle placed at the base of the tail. A ground needle is inserted on the lower back of the mice. The sciatic nerve is stimulated with a single 0.2 msec pulse at supramaximal intensity (12.9 mA). The amplitude (mV) and the latency of the response (ms) are measured. The amplitude is indicative of the number of active motor units, while distal latency reflects motor nerve conduction velocity.

The efficacy of test compounds can also be evaluated using biomarker analysis. To assess the regulation of protein biomarkers in SOD1 mice during the onset of motor impairment, samples of lumbar spinal cord (protein extracts) are applied to ProteinChip Arrays with varying surface chemical/biochemical properties and analyzed, for example, by surface enhanced laser desorption ionization time of flight mass spectrometry. Then, using integrated protein mass profile analysis methods, data is used to compare protein expression profiles of the various treatment groups. Analysis can be performed using appropriate statistical methods.

### Example 9

### Animal Model for Assessing Therapeutic Efficacy of Forms of Propofol for Treating Diabetic Neuropathy

Following an overnight fast, 8 week old male C57BL/6J mice are injected i.p. with 55 mg/kg of streptozotocin dissolved in citrate buffer (pH 5.5) for 5 days to induce diabetes. Diabetes is defined as blood glucose over 200 mg/dL. Diabetes manifests in heterozygous male B6Ins2^{Akita} mice and male and female B6-db/db and BKS-db/db mice at 8 weeks of age. B6-db/db and B6-db+ mice are maintained on either a synthetic diet (11.5% kcal derived from fat, lacking phytoestrogents) or an increased fat diet (17% kcal derived from fat). All other mice are fed standard mouse chow (12% kcal derived from fat).

Blood glucose levels are measured every 4 weeks to monitor the persistence and duration of diabetes. Following a 6 h fast, one drop of tail blood is analyzed.

Mice are placed in an acrylic holder atop a tail flick analgesia meter so that the tail is in contact with an adjustable red light emitter (range 60-170° C.). The time from activation of the beam to animal response is recorded. Hind paw analgesia is measured using the same apparatus. Mice are placed in compartments on a warm (32° C.) glass plate and allowed to habituate for 10 min. The light source is maneuvered under the hind paw and the time of activation of the beam to the time of paw withdrawal is recorded. The light source is set at 25° C. and the temperature increased to 70° C. during 10 s.

Measures of nerve conduction velocity (NCV) are performed according to procedures described in Layton et al., J Biomech 2004, 37, 879-888. Mice are anesthetized and body temperature monitored with a dermal temperature probe and maintained at 34° C. with a warming lamp. The recording/stimulating electrodes in the tail are placed 30 mm apart. For the sciatic nerve, the recording electrodes are placed in the dorsum of the foot and the stimulating electrodes at the knee and sciatic notch. For stimulation, the cathode is distal and the anode is placed along the length of the nerve, 5 mm from the cathode. The frequency band is inclusive of two, 10 Hz for muscle potential recordings and 10, 2 Hz for sensory potential recordings.

Tissues are harvested 24 weeks post induction of diabetes for biochemical analysis. To determine intraepidermal nerve fiber density (IENF), foot pads are collected from the plantar surface of the hind paw, immersed in Zamboni's fixative and processed for pan-axonal marker, PGP9.5, immunofluorescence. The number of fibers per linear millimeter of epidermis is determined. Nuclear DNA fragmentation can be measured according to the method of Russell et al., FASEB J 2002, 16, 1738-1748. The level of reactive nitrogen species can be determined using anti-nitrotyrosine immunofluorescence according the method of Ilnytska et al., Diabetes 2006, 55, 1686-1694.

Test compound can be administered and the impact of the measures of diabetic neuropathy determined.

### Example 10

### Methods of Determining Efficacy in Treating Liver Diseases

### Non-Alcoholic Steatohepatitis

A choline deficient L-amino acid (CDAA) defined diet-induced liver fibrosis animal model of NASH according to Koteish and Diehl, Semin Liver Dis 2001, 21, 89-104, can be used to assess the efficacy of a compound for treating NASH.

Male Wistar rats, 6 wks old and weighing 140-150 g are used. The total study periods are 2 and 10 weeks. Groups of rats receive a CDAA diet, a CDAA diet with administered test compound, a choline-supplemented L-amino acid-defined (CSAA) diet, or a CSAA diet with administered test compound. All groups receive the same amount of food.

In the two-week experiment, the content of triacylglycerol in the liver tissue is determined according to the method of Folch et al., J Biol Chem 1957, 226, 497-509. In all experiments, serum ALT, alkaline phosphatase (ALP), triacylglycerol (TG), hyaluronic acid, and bile acid are measured. Five-μm thick sections of the right lobe of all rat livers, fixed in 10% formalin for 24 h and embedded in paraffin, are processed for sirius red staining. α-Smooth muscle actin (αSMA) for the detection of activated stellate cells, and glutathione S-transferase placental form (GST-P) positive lesions (as preneoplastic lesions) are immunohistochemically assessed by the avidin-biotin-peroxidase complex method as described by Sakaida et al., Hepatology 1998, 28, 2201-2206. αSMA and GSTP-positive cells in the liver are quantified using microscopy. The area of sirius red positive area and αSMA-positive cells are expressed as the percentage of the total area of the specimen. The size and number of GST-P positive lesions are counted in each specimen.

Expression of type I procollagen MMP-2, MMP-13, TIMP-1, and TIMP-2 mRNA was determined by real-time PCR as described by Yoshiji et al., Hepatology 2001, 34, 745-750.

Analysis of results using a similar model are described, for example, in Kawaguchi et al., Biochem Biophys Res Commun 2004, 315, 187-195.

### Non-Alcoholic Fatty Liver Disease

Male Wistar rats weighing 300 to 350 g are used. Fatty liver is induced in the animals by choline deficient diet for four weeks. The animals are randomly divided into two groups: a control group fed with choline deficient diet plus administration of vehicle; Test Compound group fed choline deficient diet plus administration of test compound. After a period of treatment, such as for example, 4 weeks, plasma samples are collected, animals are sacrificed, and their livers collected for histological examination and lipid peroxidation analysis.

Serum alanine aminotransferase (AST), aspartate aminotransferase (ALT), cholesterol and triglycerides are analyzed by standard methods (see, e.g., Rubbo et al., Biol Chem 2002, 383, 547-552).

Fragments of liver tissue are fixed by immersion in formaldehyde saline (10%) and are processed by hematoxylin-eosin and Masson trichrome staining for histological analysis. Scharlach red fat staining is sued for more accurate evaluation of fatty change. Histological variables are blindly semiquantitated from 0 to 4+ with respect to macro and microvacuolar fatty change, the zonal distribution of fatty change, foci of necrosis, portal and perivenular fibrosis as well as inflammatory infiltrate with zonal distribution.

Samples of liver homogenates are extracted with a mixture of acetonitrile:hexane (4:10, v/v). The contents are vortexed for 2 min and centrifuged at 2,500 rpm for 10 min for phase separation. The hexane phase containing chloesteryl ester derived hydroperoxides (LOOH) is collected and evaporated under nitrogen. The residue is dissolved in methanol:butanol (2:1, v/v), filtered and analyzed by HPLC. Results are expressed as nmol of lipid hydroperoxides/mg of protein.

### Example 11

### Methods of Determining Efficacy in Treating Pulmonary Diseases

### Asthma

Male rats weighing 220-300 g are actively sensitized by intraperitoneal injection of 1 mL of a suspension of 1 mg ovalbumin and 100 mg of aluminum hydroxide [Al(OH)₃] in 0.9% (wt/vol) saline for three consecutive days. The sensitized animals are used for experiments 21 days after the initial injection. This procedure has been shown to result in the development of immunoglobulin E-type antibody (Elwood et al., Int Arch Allergy Immunol 1992, 99, 91-97).

Animals are randomly distributed into four groups. The untreated groups are a negative control (Group A) consisting of sensitized animals receiving drug vehicle and exposed to aerosol saline, and a positive control (Group B) comprising sensitized animals subsequently exposed to aerosol antigen and receiving drug vehicle. Group C comprised the sensitized animals treated with test compound and challenged with antigen. An additional group of sensitized rats receive test compound but are challenged with saline instead of antigen.

The following procedure is used to assess the effects of test compound on antigen-induced acute bronchoconstriction. Animals are anesthetized and instrumented as described by Advenier et al., Br J. Pharmacol 1972, 44, 642-50. The airflow, transpulmonary pressure, and arterial blood pressure are measured and the lung resistance calculated according to Amdur and Mead, Am J Physiol 1958, 192, 364-368. After 10 min stabilization, animals are challenged with inhaled antigen (100 mg/mL, 5 min) as described by Olivenstein et al., Pulm Pharmacol Ther 1997, 10, 223-230).

The following procedure is used to assess the effects of test compound on airway hyperresponsiveness and eosinophil infiltration. Sensitized conscious rats are exposed to antigen aerosol in a clear plastic chamber, which is connected to the output of a nebulizer. The nebulizer out put is approximately 8-10 mL/h. The duration of the antigen challenge is 60 min. The time course of airway hyper-reactivity in antigen-exposed rats has been examined (Elwood et al., Int Arch Allergy Immunol 1992, 99, 91-97) and the response after 24 h is selected accordingly. Twenty-four hours after exposure to the aerosol, airway reactivity is determined form dose-response curves to 5-hydroxytryptamine (5-HT), administered (6.25, 12.5, 25, 50, and 100 μg/mL) to animals anesthetized and instrumented as previously. 5-HT has been used in rats since it provides a reproducible bronchoconstrictor response and does not require pretreatment with propranolol (Carvalho et al., Exp Lung Res 1999, 25, 303-316).

After measurement of airway reactivity, animals are killed by an overdose of urethane. Bronchoalveolar cells are collected in two successive lavages using 6 mL aliquots of sterile saline and heparin 10 IU/mL at room temperature injected and recovered through a tracheal cannula. Cell pellets are obtained by low-speed centrifugation. Total cell counts are made using a haemocytometer. Differential cell counts are determined from cytospin preparations by counting 300 cells stained with May-Grunwald-Giemsa, and the results expressed as cell number/mL.

The following procedure is used to assess the effects of test compound on microvascular leakage after antigen challenge. Animals are prepared as described by Olivenstein et al., Pulm Pharmacol Ther 1997, 10, 223-230, and anesthetized and instrumented as previously described. After 10 min stabilizatiion, the animals receive an injection of Evans blue dye (30 mg/kg, i.v.) and 1 min later, aerosol antigen is administered (100 mg/mL, 5 min). Five min after antigen inhalation the animals are hyperinflated with twice the tidal volume by manually blocking the outflow of the ventilator. The animals are disconnected from the ventilator and subjected to bronchoalveolar lavage (two aliquots of 1 mL saline) for measurement of Evans blue dye extravasation into the airway lumen. Taurine levels are measured in supernatant of bronchoalveolar lavage fluid by fluorimetery.

### Pulmonary Fibrosis

Bleomycin (3 mg/kg) is administred to male C57BL/6 (8-10 wk old) mice. On days 3, 7, and 14 following bleomycin treatment, the animals are killed and the lungs removed. Animals are allocated to four groups, as follows: (1) saline and vehicle; (2) saline and test compound; (3) bleomycin and vehicle; and (4) bleomycin and test compound. The right lung is fixed in 10% buffered formalin, and stained with hematoxylin, eosin, and Masson's trichrome. Histologic grading of fibrosis is performed using a blinded semiquantitative scoring system for extent and severity of fibrosis in lung parenchyma. Severity of fibrosis is scored according to the method of Ashcroft et al., J Clin Pathol 1988, 41, 467-470. To assay for collagen, the left lung is homogenized and the collagen content determined.

For immunochemistry, lung tissues are prepared according to Sato et al., Am J Pathol 1986, 125, 431-435. Sections taken from paraffin-embedded samples are immunostained for epidermal growth factor receptor (EGFR) and phosphorylated EGFR by the labeled streptavidin-biotin method as described by Pfeiffer et al., Appl Immunohistochem Mol Morphol 1996, 4, 135-138. To evaluate fibroblast proliferation and expression of EGFR on fibroblasts, lungs are double-immunostained for fibroblast-specific marker S100A4 (Spurgeon et al., Am J Physiol Renal Physiol 2005, 288, F568-F577) and EGFR. For the representative samples, immunofluorescent double-staining for S100A4 and EGFR is also performed. For a semiquantitative analysis of receptor expression, more than 500 cells per immunostained section are observed to count positive cells. The labeling index is calculated as follows: labeling index (%)=positive cells/all counted cells×100.

Data is analyzed using appropriate statistical methods.

Efficacy of the test compound for treating pulmonary fibrosis is indicated by a reduced EGFR phosphorylation, reduced collagen content, reduced fibrosis score, and reduced immunohistochemical labeling index compared to control.

Finally, it should be noted that there are alternative ways of implementing the disclosures contained herein. Accordingly, the present embodiments are to be considered as illustrative and not restrictive, and the claims are not to be limited to the details given herein, but may be modified within the scope and equivalents thereof.

What is claimed is:

1. A method of treating a disease associated with oxidative stress in a patient comprising orally administering to a patient in need of such treatment a therapeutically effective amount of at least one form of propofol that provides a high oral bioavailability of propofol.

2. The method of claim 1, wherein the form of propofol is a propofol prodrug and is chosen from a compound of Formula (I), Formula (II), Formula (III), Formula (IV), a pharmaceutically acceptable salt of any of the foregoing, and a pharmaceutically acceptable solvate of any of the foregoing.

3. The method of claim 2, wherein the propofol prodrug is (S)-2-amino-3-(2,6-diisopropylphenoxycarbonyloxy)-propanoic acid, a pharmaceutically acceptable salt thereof, or a pharmaceutically acceptable solvate of any of the foregoing.

4. The method of claim 1, comprising maintaining a propofol concentration in the blood of the patient ranging from about 10 ng/mL to about 5,000 ng/mL for at least about 4 hours following oral administration of the form of propofol to the patient.

5. The method of claim 1, comprising maintaining a propofol concentration in the blood of the patient ranging from about 10 ng/mL to about 2,000 ng/mL for at least about 4 hours following oral administration of the form of propofol to the patient.

6. The method of claim 1, wherein the therapeutically effective amount is less than an amount that causes moderate sedation in the patient.

7. The method of claim 1, wherein the disease associated with oxidative stress is chosen from a metabolic disease, a cardiovascular disease, a neurological disease, a liver disease, and a pulmonary disease.

8. The method of claim 7, wherein the metabolic disease is chosen from diabetes mellitus type I, diabetes mellitus type II, metabolic syndrome, hypertension, obesity, and dyslipidemia.

9. The method of claim 7, wherein the cardiovascular disease is chosen from congestive heart failure, myocardial infarction, pulmonary hypertension, hypertrophic cardiomyopathy, arrhythmias, aoritic stenosis, angina pectoris, cardiac arrhythmia, ischemic stroke, ischemic cardiomyopathy, and stroke.

10. The method of claim 7, wherein the neurological disease is chosen from Parkinson's disease, Alzheimer's disease, amyotrophic lateral sclerosis, multiple sclerosis, and diabetic neuropathy.

11. The method of claim 7, wherein the liver disease is chosen from alcoholic liver disease, chronic viral hepatitis, autoimmune liver diseases, and non-alcoholic steatohepatitis, and non-alcoholic fatty liver disease.

12. The method of claim 7, wherein the pulmonary disease is chosen from asthma, chronic obstructive pulmonary fibrosis, idiopathic pulmonary fibrosis, pulmonary fibrosis, acute respiratory distress syndrome, interstitial lung diseases, bronchopulmonary dysplasia, and cystic fibrosis.
